(19) United States
(12) Patent Application Publication
(10) Pub. No.: US 2026/0256753 A1
(43) Pub. Date: Sep. 3, 2026

(54) USE OF AN OREXIN 2 RECEPTOR AGONIST FOR IMPROVING RESPIRATORY FUNCTION DURING SLEEP

(71) Applicant: TAKEDA PHARMACEUTICAL COMPANY LIMITED, Osaka (JP)

(72) Inventors: Haruhide KIMURA, Kanagawa (JP); Ryuji YAMADA, Kanagawa (JP)

(21) Appl. No.: 19/489,193

(22) PCT Filed: May 31, 2024
(86) PCT No.: PCT/IB2024/055361

§ 371 Date: Dec. 1, 2025

Related U.S. Application Data
(60) Provisional application No. 63/505,795, filed on Jun. 2, 2023.

(51) Int. Cl. A61K 31/4418; A61K 9/00; A61P 11/00

(52) U.S. Cl. CPC A61K 31/4418; A61K 9/0053; A61P 11/00

(57) ABSTRACT

Disclosed are uses of an orexin type 2 receptor (OX2R) agonist at non-awakening concentrations to treat sleep apnea. Also disclosed are compositions and methods for administering an OX2R agonist to a subject (e.g., mammal) in need thereof at a dose that provides a blood plasma concentration of the OX2R agonist that is at or below the maximum non-awakening blood plasma concentration of the OX2R agonist. Also disclosed are compositions and methods for treating sleep apnea in a subject in need thereof by maintaining the blood plasma concentration of an OX2R agonist following administration which is at or below the maximum non-awakening concentration of the OX2R agonist.

### BACKGROUND

Respiration is a critical process involving exchange of oxygen and carbon dioxide in the lung. The efficiency of this exchange is dependent on ventilation, which is the movement of air into the lungs via inhalation and exhalation. Ventilation needs precise regulation of the muscles of respiration and airway including the diaphragm and genioglossus muscle (the main upper airway dilator muscle). Descending movement of the diaphragm into the abdominal cavity increases lung volume and induces inspiration (M. de Carvalho, et al., Diaphragmatic Neurophysiology and Respiratory Markers in ALS, Frontiers in Neurology 10 (2019)), and anterior movement of the tongue by the genioglossus muscle widens the oropharyngeal airway (A. S. Jordan, D. P. White, Pharyngeal motor control and the pathogenesis of obstructive sleep apnea, Respir Physiol Neurobiol 160 (1) (2008) 1-7). Neurons in the pre-Bötzinger complex located in the ventrolateral medulla regulate the coordinated activity of the diaphragm and genioglossus muscle through controlling the activity of phrenic motoneurons in the cervical spinal cord and hypoglossal motoneurons in dorsomedial medulla, respectively (J. C. Smith, A. P. Abdala, A. Borgmann, I. A. Rybak, J. F. Paton, Brainstem respiratory networks: building blocks and microcircuits, Trends Neurosci 36 (3) (2013), 152-62) and are critical for the generation of inspiratory air flow.

Dysregulation of the respiratory system is associated with various medical conditions, such as opioid-induced respiratory depression after surgery (S. Ayad, A. K. Khanna, S. U. Iqbal, N. Singla, Characterisation and monitoring of postoperative respiratory depression: current approaches and future considerations, Br J Anaesth 123 (3) (2019), 378-391), opioid overdose (M. Nagappa, T. N. Weingarten, G. Montandon, J. Sprung, F. Chung, Opioids, respiratory depression, and sleep-disordered breathing, Best Pract Res Clin Anaesthesiol 31 (4) (2017) 469-485), and sleep apnea (obstructive, mixed and central types) (J. A. Dempsey, S. C. Veasey, B. J. Morgan, C. P. O'Donnell, Pathophysiology of sleep apnea, Physiol Rev 90 (1) (2010) 47-112), which is characterized by intermittent pauses in breathing during sleep causing hypoxia and cortical arousals. After surgery, opioids are given to manage pain, however, their usage can be associated with severe adverse events, including respiratory and CNS depression (C. J. van Dam, M. H. Algera, E. Olofsen, L. Aarts, T. Smith, M. van Velzen, E. Sarton, M. Niesters, A. Dahan, Opioid utility function: methods and implications, Ann Palliat Med 9 (2) (2020) 528-536). The opioid antagonist, naloxone, reduces these adverse events however, it also suppresses the opioid analgesia. Moreover, because of life-threatening cardiovascular side effects, use of naloxone must be carefully considered (A. Dahan, L. Aarts, T. W. Smith, Incidence, Reversal, and Prevention of Opioid-induced Respiratory Depression, Anesthesiology 112 (1) (2010) 226-38). Thus, drugs that suppress opioid-induced respiratory depression (OIRD) without compromising analgesia are required (M. Z. Imam, A. Kuo, M. T. Smith, Countering opioid-induced respiratory depression by non-opioids that are respiratory stimulants, F1000Res 9 (2020)).

There are several types of sleep apnea: central sleep apnea (CSA), obstructive sleep apnea (OSA), mixed apnea and complex sleep apnea (T. I. Morgenthaler, V. Kagramanov, V. Hanak, P. A. Decker, Complex sleep apnea syndrome: is it a unique clinical syndrome?, Sleep 29 (9) (2006) 1203-9). CSA is associated with decreased respiratory signals from the brainstem respiratory center to the diaphragm (J. A. Dempsey, et al.). OSA is associated with the obstruction of the upper airway (J. A. Dempsey, et al.). Complex apnea features patients with obstructive apneas who develop central apneas after treatment. Continuous positive airway pressure (CPAP), the gold standard treatment, can maintain an airway in patients with sleep apnea by delivering air through a mask on the nose and/or mouth and is widely used to treat both CSA and OSA to physically splint the upper airway open. However, CPAP has an issue of poor adherence with reduced nighttime use or total elimination of treatment (A. M. Sawyer, N. S. Gooneratne, C. L. Marcus, D. Ofer, K. C. Richards, T. E. Weaver, A systematic review of CPAP adherence across age groups: clinical and empiric insights for developing CPAP adherence interventions, Sleep Med Rev 15 (6) (2011) 343-56). Although several compounds are reported to improve OSA (D. W. Carley, B. Prasad, K. J. Reid, R. Malkani, H. Attarian, S. M. Abbott, B. Vern, H. Xie, C. Yuan, P. C. Zee, Pharmacotherapy of Apnea by Cannabimimetic Enhancement, the PACE Clinical Trial: Effects of Dronabinol in Obstructive Sleep Apnea, Sleep 41 (1) (2018); L. Taranto-Montemurro, L. Messineo, S. A. Sands, A. Azarbarzin, M. Marques, B. A. Edwards, D. J. Eckert, D. P. White, A. Wellman, The Combination of Atomoxetine and Oxybutynin Greatly Reduces Obstructive Sleep Apnea Severity. A Randomized, Placebo-controlled, Double-Blind Crossover Trial, Am J Respir Crit Care Med 199 (10) (2019) 1267-1276), currently no drugs are approved by the US Food and Drug Administration for the treatment of CSA and OSA. Therefore, novel pharmaceutical approaches for treating sleep apnea are needed.

### SUMMARY

In one aspect, the disclosure provides a method for improving respiratory function during sleep in a subject in need thereof, comprising administering to the subject an orexin type 2 receptor agonist.

In some embodiments, administering the orexin type 2 receptor agonist regulates inspiratory air flow in the subject.

In some embodiments, the orexin type 2 receptor agonist is administered in an amount that is effective to improve respiratory function while not providing an arousal response or wakefulness in the subject.

In some embodiments, the arousal response or wakefulness is determined by measuring sleep latency in one or more subjects diagnosed with sleep apnea.

In some embodiments, the subject has sleep apnea.

In some embodiments, the sleep apnea is selected from obstructive sleep apnea (OSA), treatment emergent sleep apnea, and central sleep apnea (CSA).

In some embodiments, the sleep apnea is obstructive sleep apnea (OSA).

In some embodiments, administration of the OX2R agonist reduces the Apnea Hypopnea Index (AHI) in the subject. In some embodiments, after administration, an Apnea Hypopnea Index (AHI) in the subject is reduced relative to the absence of administering the orexin type 2 receptor agonist.

In some embodiments, administration of the OX2R agonist reduces the hypoxic burden in the subject. In some embodiments, after administration, an hypoxic burden in the subject is reduced relative to the absence of administering the orexin type 2 receptor agonist.

In some embodiments, the administration of the OX2R agonist provides a blood plasma concentration of the OX2R agonist at or below a maximum non-awakening plasma concentration of the agonist over a dosing interval.

In some embodiments, the blood plasma concentration of the OX2R agonist is selected by: i) determining the non-awakening plasma concentration of the OX2R agonist that does not provide an arousal response or wakefulness in a human; and ii) determining a dose of the OX2R agonist that will provide a blood plasma concentration which is at or below the maximum non-awakening plasma concentration.

In some embodiments, administration of the OX2R agonist provides a blood plasma concentration of the agonist that is bioequivalent to a blood plasma concentration of from about 5 ng/ml to about 100 ng/ml of methyl 3-((methylsulfonyl)amino)-2-(((4-phenylcyclohexyl)oxy)methyl) piperidine-1-carboxylate over a dosing interval after administering to the subject the methyl 3-((methylsulfonyl)amino)-2-(((4-phenylcyclohexyl)oxy)methyl) piperidine-1-carboxylate, or a pharmaceutically acceptable salt thereof.

In some embodiments, the blood plasma concentration of the methyl 3-((methylsulfonyl)amino)-2-(((4-phenylcyclohexyl)oxy)methyl) piperidine-1-carboxylate is about 10 ng/mL to about 30 ng/mL.

In some embodiments, the OX2R agonist is administered orally, intravenously, subcutaneously, transdermally, or transmucosally. In some embodiments, the OX2R agonist is administered orally.

In some embodiments, the OX2R agonist is administered once per day. In some embodiments, the OX2R agonist is administered from about 5 minutes to about 5 hours before the subject's bedtime.

In some embodiments, the orexin type 2 receptor agonist is a compound represented by the formula (I):

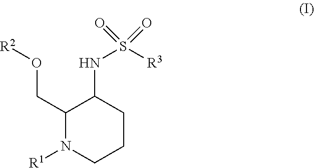

wherein
R¹ is
   (1) a hydrogen atom,
   (2) a C₁₋₆ alkyl-carbonyl group optionally substituted by 1 to 7 substituents selected from
      (i) a halogen atom, (ii) a cyano group, (iii) a hydroxy group, (iv) a C₃₋₁₀ cycloalkyl group, (v) a C₁₋₆ alkoxy group, (vi) a C₆₋₁₄ aryl group, (vii) a C₆₋₁₄ aryloxy group, (viii) a pyrazolyl group, a thiazolyl group, a pyrimidinyl group or a pyridazinyl group, each of which is optionally substituted by an oxo group, (ix) a pyrazolyloxy group optionally substituted by 1 to 3 C₁₋₆ alkyl groups, (x) a C₁₋₆ alkyl-carbonyl group, (xi) a C₁₋₆ alkoxy-carbonyl group, (xii) a C₁₋₆ alkyl-carbonyloxy group, (xiii) a C₁₋₆ alkylsulfonyl group, (xiv) a mono- or di-C₁₋₆ alkylamino group, (xv) a C₁₋₆ alkyl-carbonylamino group and (xvi) a (C₁₋₆ alkyl) (C₁₋₆ alkyl-carbonyl)amino group,
   (3) a C₃₋₁₀ cycloalkyl-carbonyl group optionally substituted by 1 to 3 substituents selected from a halogen atom, a cyano group, a hydroxy group, an oxo group and a C₁₋₆ alkyl group,
   (4) a C₁₋₆ alkoxy-carbonyl group optionally substituted by 1 to 6 substituents selected from deuterium, a halogen atom and a C₆₋₁₄ aryl group,
   (5) a C₃₋₁₀ cycloalkyloxy-carbonyl group optionally substituted by 1 to 3 substituents selected from a C₁₋₆ alkyl group,
   (6) a C₆₋₁₄ aryl-carbonyl group optionally substituted by 1 to 3 substituents selected from a halogen atom and a C₆₋₁₄ aryl group,
   (7) a C₆₋₁₄ aryloxy-carbonyl group,
   (8) a furylcarbonyl group, a thienylcarbonyl group, a pyrazolylcarbonyl group, an isoxazolylcarbonyl group or a pyridylcarbonyl group, each of which is optionally substituted by 1 to 3 substituents selected from a C₁₋₆ alkyl group,
   (9) an azetidinylcarbonyl group, an oxetanylcarbonyl group, a pyrrolidinylcarbonyl group, a tetrahydrofuranylcarbonyl group, a tetrahydropyranylcarbonyl group or a morpholinylcarbonyl group, each of which is optionally substituted by 1 to 3 substituents selected from an oxo group, a C₁-6 alkyl-carbonyl group, a C₁-6 alkoxy-carbonyl group and a C₁₋₆ alkylsulfonyl group,
   (10) a mono- or di-C₁₋₆ alkyl-carbamoyl group optionally substituted by 1 to 3 substituents selected from a halogen atom, a cyano group, a hydroxy group and a C₁₋₆ alkoxy group,
   (11) a mono- or di-C₃₋₁₀ cycloalkyl-carbamoyl group,
   (12) a mono- or di-C₆₋₁₄ aryl-carbamoyl group,
   (13) a C₁₋₆ alkylsulfonyl group,
   (14) a C₃₋₁₀ cycloalkylsulfonyl group,
   (15) a C₆₋₁₄ arylsulfonyl group optionally substituted by 1 to 3 halogen atoms,
   (16) a thienylsulfonyl group, a pyrazolylsulfonyl group, an imidazolylsulfonyl group, a pyridylsulfonyl group or a dihydrochromenylsulfonyl group, each of which is optionally substituted by 1 to 3 substituents selected from a C₁₋₆ alkyl group,
   (17) a mono- or di-C₁₋₆ alkyl-sulfamoyl group or
   (18) a C₁₋₆ alkyl-carbonyl-carbonyl group;
R² is a C₃₋₆ cycloalkyl group, a pyrrolidinyl group, a piperidinyl group or a dioxanyl group, each of which is optionally substituted by 1 to 3 substituents selected from
   (1) deuterium,
   (2) a halogen atom,
   (3) a hydroxy group,
   (4) a C₁₋₆ alkyl group optionally substituted by 1 to 3 substituents selected from a halogen atom and a C₆₋₁₄ aryl group,
   (5) a C₃₋₁₀ cycloalkyl group,
   (6) a C₁₋₆ alkoxy group optionally substituted by a C₃₋₁₀ cycloalkyl group,
   (7) a C₆₋₁₄ aryl group optionally substituted by 1 to 3 substituents selected from a halogen atom, a cyano group, a C₁-6 alkyl group optionally substituted by 1 to 3 halogen atoms, a C₁₋₆ alkoxy group optionally substituted by 1 to 3 halogen atoms and a hydroxy group,
   (8) a C₆₋₁₄ aryloxy group,
   (9) a tri-C₁₋₆ alkylsilyloxy group,
   (10) a pyrazolyl group, a thiazolyl group, a pyridyl group, a pyrimidinyl group, a quinazolinyl group, a benzothiazolyl group or an isoquinolinyl group, each of which is optionally substituted by 1 to 3 substituents selected from a halogen atom, a C₁₋₆ alkyl group and a C₁₋₆ alkoxy group, and
   (11) a C₆₋₁₄ aryl-carbonyl group; and
R³ is a C₁₋₆ alkyl group, or a mono- or di-C₁₋₆ alkylamino group,
or a pharmaceutically acceptable salt thereof.

In some embodiments, R¹ is
(1) a hydrogen atom,
(2) a C₁₋₆ alkyl-carbonyl group optionally substituted by a hydroxy group,
(3) a cyclopropanecarbonyl group,
(4) a C₁₋₆ alkoxy-carbonyl group or
(5) a mono- or di-C₁₋₆ alkyl-carbamoyl group;
R² is
(A) a cyclohexyl group optionally substituted by 1 to 3 substituents selected from
   (1) a C₁₋₆ alkyl group and
   (2) a phenyl group optionally substituted by 1 to 3 substituents selected from a halogen atom, a C₁₋₆ alkyl group optionally substituted by 1 to 3 halogen atoms and a C₁₋₆ alkoxy group or
(B) a piperidinyl group optionally substituted by 1 to 3 pyrimidinyl groups; and
R³ is a C₁₋₆ alkyl group or a di-C₁₋₆ alkylamino group,
or a pharmaceutically acceptable salt thereof.

In some embodiments, the orexin type 2 receptor agonist is selected from Methyl (2R,3S)-3-((methylsulfonyl)amino)-2-(((cis-4-phenylcyclohexyl)oxy)methyl) piperidine-1-carboxylate; N-((2R,3S)-1-glycoloyl-2-(((cis-4-(2,3,6-trifluorophenyl)cyclohexyl)oxy)methyl) piperidin-3-yl) methanesulfonamide; and (2R,3S)—N-ethyl-2-(((cis-4-isopropylcyclohexyl)oxy)methyl)-3-((methylsulfonyl)amino) piperidine-1-carboxamide; or a pharmaceutically acceptable salt thereof.

In some embodiments, the orexin type 2 receptor agonist is selected from methyl (2R,3S)-3-((methylsulfonyl)amino)-2-(((cis-4-phenylcyclohexyl)oxy)methyl) piperidine-1-carboxylate, or a pharmaceutically acceptable salt thereof.

In some embodiments, the orexin type 2 receptor agonist is selected from N-((2S,3S)-1-(2-hydroxy-2-methylpropanoyl)-2-((2,3′,5′-trifluorobiphenyl-3-yl)methyl) pyrrolidin-3-yl) methanesulfonamide; N-((2S,3S)-2-((2,3′-difluorobiphenyl-3-yl)methyl)-1-(2-hydroxy-2-methylpropanoyl) pyrrolidin-3-yl) ethanesulfonamide; methyl (2R,3S)-3-((methylsulfonyl)amino)-2-(((cis-4-phenylcyclohexyl)oxy)methyl) piperidine- 1-carboxylate; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}methanesulfonamide; 4-(5-cyclopropyl-1,2,4-oxadiazol-3-yl)-N-{(1R,6S)-2,2-difluoro-6-[4-(propan-2-yl) piperazin-1-yl]cyclohexyl}-4-methylpiperidine-1-carboxamide; N-{(1R,6S)-2,2-difluoro-6-[4-(propan-2-yl) piperazin-1-yl]cyclohexyl}-4-{5-[(1S,2S)-2-fluorocyclopropyl]-1,2,4-oxadiazol-3-yl}-4-methylpiperidine-1-carboxamide; (2R)-2-cyclopropyl-2-{(1R,3S,5S)-3-[(3S,4R)-1-(5-fluoropyrimidin-2-yl)-3-methoxypiperidin-4-yl]-8-azabicyclo[3.2.1]octan-8-yl}acetamide; (R)-2-((1R,3S,5S)-3-((3S,4R)-1-(5-fluoropyrimidin-2-yl)-3-methoxypiperidin-4-yl)-8-azabicyclo[3.2.1]octan-8-yl)-3-methylbutaneamide; (R)-2-((1R,3S,5S)-3-((3S,4R)-1-(5-chloropyrimidin-2-yl)-3-ethoxypiperidin-4-yl)-8-azabicyclo[3.2.1]octan-8-yl)-2-cyclopropyl acetamide; (R)-2-cyclopropyl-2-((1R,3S,5S)-3-((2S,4S)-1-(5-fluoropyrimidin-2-yl)-2-methylpiperidin-4-yl)-8-azabicyclo[3.2.1]octan-8-yl) acetamide; and N-((2¹S,2⁴S,5²R,5³S)-6-oxo-3,8-dioxa-1 (2,3)-pyrazina-5 (2,1)-piperidina-2 (1,4)-cyclohexanacyclooctaphane-5³-yl) methanesulfonamide; or a pharmaceutically acceptable salt thereof.

In some embodiments, the orexin type 2 receptor agonist is a compound represented by the formula (II):

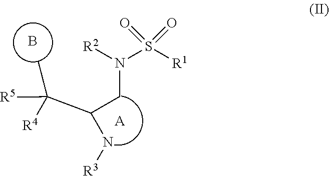

wherein
R¹ is
   (1) a C₁₋₆ alkyl group optionally substituted by 1 to 3 substituents selected from
      (a) a halogen atom, and
      (b) a C₁₋₆ alkoxy group,
   (2) a C₃₋₆ cycloalkyl group optionally substituted by 1 to 3 halogen atoms, or
   (3) a mono- or di-C₁₋₆ alkylamino group;
R² is a hydrogen atom;
R³ is
   (1) a C₁₋₆ alkoxy-carbonyl group,
   (2) a C₁₋₆ alkyl-carbonyl group optionally substituted by 1 to 3 hydroxy groups,
   (3) a mono- or di-C₁₋₆ alkyl-carbamoyl group,
   (4) a N—C₁₋₆ alkyl-N—C₁₋₆ alkoxy-carbamoyl group,
   (5) a C₃₋₆ cycloalkyl-carbonyl group (the C₃₋₆ cycloalkyl in the C₃₋₆ cycloalkyl-carbonyl group may be a bridged ring group) optionally substituted by 1 to 3 substituents selected from
      (a) a halogen atom,
      (b) a C₁₋₆ alkyl group optionally substituted by 1 to 3 halogen atoms,
      (c) a hydroxy group,
      (d) a C₁₋₆ alkoxy group, and
      (e) a cyano group,
   (6) an oxetanylcarbonyl group,
   (7) an azetidinylcarbonyl group optionally substituted by 1 to 3 substituents selected from
      (a) a halogen atom, and
      (b) a C₁₋₆ alkyl group, or
   (8) a 5-azaspiro[2.3]hexylcarbonyl group;
R⁴ and R⁵ are both hydrogen atoms;
Ring A is
   (1) a pyrrolidine ring, or
   (2) a piperidine ring; and
Ring B is
   (1) a benzene ring further substituted by one phenyl group optionally substituted by 1 to 3 substituents selected from
      (i) a halogen atom, and
      (ii) a C₁₋₆ alkyl group, and
optionally further substituted by one halogen atom,
   (2) a pyridine ring further substituted by one phenyl group optionally substituted by 1 to 3 halogen atoms,
   (3) a thiazole ring further substituted by one phenyl group optionally substituted by 1 to 3 halogen atoms, or
   (4) a piperidine ring further substituted by one phenyl group;
or a pharmaceutically acceptable salt thereof.

In some embodiments, the orexin type 2 receptor agonist is N-((2S,3S)-1-(2-hydroxy-2-methylpropanoyl)-2-((2,3′,5′-trifluorobiphenyl-3-yl)methyl) pyrrolidin-3-yl) methanesulfonamide, or a pharmaceutically acceptable salt thereof.

In some embodiments, the orexin type 2 receptor agonist is N-((2S,3S)-2-((2,3′-difluorobiphenyl-3-yl)methyl)-1-(2-hydroxy-2-methylpropanoyl) pyrrolidin-3-yl) ethanesulfonamide, or a pharmaceutically acceptable salt thereof.

In some embodiments, the orexin type 2 receptor agonist is a compound represented by the formula (III):

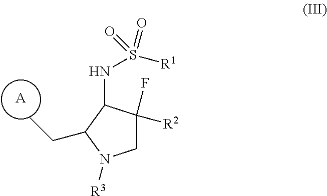

wherein
R¹ is
   (1) a C₁₋₆ alkyl group,
   (2) a mono- or di-C₁₋₆ alkylamino group, or
   (3) a C₃₋₆ cycloalkyl group;
R² is
   (1) a hydrogen atom,
   (2) a fluorine atom, or
   (3) a C₁₋₆ alkyl group;
R³ is
   (1) a C₁₋₆ alkyl-carbonyl group optionally substituted by 1 to 3 substituents selected from
      (a) a halogen atom,
      (b) a hydroxy group, and
      (c) a cyano group,
   (2) a C₁₋₆ alkoxy-carbonyl group,
   (3) a C₃₋₁₀ cycloalkyl-carbonyl group (the C₃₋₁₀ cycloalkyl moiety of the C₃₋₁₀ cycloalkyl-carbonyl group is optionally bridged) optionally substituted by 1 to 3 substituents selected from
      (a) a halogen atom,
      (b) a hydroxy group,
      (c) a cyano group, and
      (d) a C₁₋₆ alkyl group,
   (4) a 3- to 14-membered non-aromatic heterocyclylcarbonyl group optionally substituted by 1 to 3 substituents selected from
      (a) a halogen atom,
      (b) a hydroxy group, and
      (c) a C₁₋₆ alkyl group,
   (5) a mono- or di-C₁₋₆ alkyl-carbamoyl group, or
   (6) a N—C₁₋₆ alkyl-N—C₁₋₆ alkoxy-carbamoyl group; and
Ring A is
   (1) a benzene ring optionally substituted by one substituent selected from
      (a) a C₆₋₁₄ aryl group optionally substituted by 1 to 3 substituents selected from
         (i) a halogen atom,
         (ii) an optionally halogenated C₁₋₆ alkyl group, and
         (iii) an optionally halogenated C₁₋₆ alkoxy group, and
      (b) a 5- to 14-membered aromatic heterocyclic group optionally substituted by 1 to 3 substituents selected from
         (i) a C₁₋₆ alkyl group, and
         (ii) a C₁₋₆ alkoxy group, and
optionally further substituted by 1 to 3 halogen atoms, or
   (2) a 5- or 6-membered aromatic heterocycle further substituted by one C₆₋₁₄ aryl group optionally substituted by 1 to 3 halogen atoms;
or a pharmaceutically acceptable salt thereof.

In some embodiments, the orexin type 2 receptor agonist is N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}ethanesulfonamide, or a pharmaceutically acceptable salt thereof.

In some embodiments, the orexin type 2 receptor agonist is N-((2S,3R)-4,4-difluoro-1-(2-hydroxy-2-methylpropanoyl)-2-((2,3′,5′-trifluoro-[1,1′-biphenyl]-3-yl)methyl) pyrrolidin-3-yl) methanesulfonamide, or a pharmaceutically acceptable salt thereof.

In some embodiments, the orexin type 2 receptor agonist is selected from N′-{(2S,3R,4S)-1-(azetidine-1-carbonyl)-4-fluoro-2-[(2-fluoro-3 methyl[1,1′-biphenyl]-3-yl)methyl]pyrrolidin-3-yl}-N,N-dimethyl sulfuric diamide; N-[(2S,3R)-2-[(2,3′-difluoro[1,1′-biphenyl]-3-yl)methyl]-4,4-difluoro-1-(2-methylpropanoyl) pyrrolidin-3-yl]ethanesulfonamide; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}ethanesulfonamide; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}methanesulfonamide; N-{(2S,3R)-1-(bicyclo[1.1.1]pentane-1-carbonyl)-4,4-difluoro-2-[(2,3′,5′-trifluoro[1,1′-biphenyl]-3-yl)methyl]pyrrolidin-3-yl}methanesulfonamide; N-{(2S,3R)-1-(cyclopropanecarbonyl)-4,4-difluoro-2-[(2,3′,5′-trifluoro[1,1′-biphenyl]-3-yl)methyl]pyrrolidin-3-yl}ethanesulfonamide; N-{(2S,3R)-4,4-difluoro-1-((1S,3R)-3-fluorocyclobutane-1-carbonyl)-2-[(2,3′,5′-trifluoro[1,1′-biphenyl]-3-yl)methyl]pyrrolidin-3-yl}ethanesulfonamide; N-{(2S,3R)-4,4-difluoro-1-((1S,3R)-3-fluorocyclobutane-1-carbonyl)-2-[(2,3′,5′-trifluoro[1,1′-biphenyl]-3-yl)methyl]pyrrolidin-3-yl}methanesulfonamide; N′-{(2S,3R)-1-(azetidine-1-carbonyl)-4,4-difluoro-2-[(2-fluoro-3′-methyl[1,1′-biphenyl]-3-yl)methyl]pyrrolidin-3-yl}-N,N-dimethylsulfuric diamide; or a pharmaceutically acceptable salt thereof.

In another aspect, the invention provides a use of an orexin type 2 receptor agonist for the manufacture of an medicament for improving respiratory function during sleep. In another aspect, the invention provides an orexin type 2 receptor agonist for use in the improvement of respiratory function during sleep.

### BRIEF DESCRIPTION OF THE DRAWINGS

FIG. 1 shows how Compound A, Compound B, and Compound C activated neurons in the pre-Bötzinger complex in rat medullary slices. Effects of Compound A on burst frequency. N=2-11. Effects of Compound B on burst frequency. N=7-21. Effects of Compound C on burst frequency. N=7-21. The neuronal activities were recorded in the presence of vehicle followed by the application of drugs. Mean values in burst frequency during the last 1-2 min in the presence of vehicle were used as control values, and those during the last 1-2 min with stimulation by each concentration of drugs were used to calculate percent changes from control values. Data are presented as mean±SEM.

FIG. 2 shows how Compound A, Compound B, and Compound C activated phrenic motoneurons in rat isolated brainstem-spinal cord preparations. Effects of Compound A on the burst frequency. N=5-6. Effects of Compound B on burst frequency. N=4-8. Effects of Compound C on the burst frequency. Curve fitting was not implemented because only 2 concentrations of Compound C were tested. N=4-5. The neuronal activities were recorded in the presence of vehicle followed by the application of drugs. Mean values in burst frequency during the last 2 min in the presence of vehicle were used as control values, and those during the last 2 min of drug perfusion were used to calculate percent changes from control values. Data are presented as mean±SEM.

FIG. 3 shows how Compound A, Compound B, and Compound C activated hypoglossal motoneurons in rat medullary slices. Effects of Compound A on burst frequency. N=5-9. Effects of Compound B on burst frequency. N=4-15. Effects of Compound C on burst frequency. N=20-21. The neuronal activities were recorded in the presence of vehicle followed by the application of drugs. Mean values in burst frequency during the last 1-2 min in the presence of vehicle were used as control values, and those during the last 1-2 min with stimulation by each concentration of drugs were used to calculate percent changes from control values. Data are presented as mean±SEM.

FIG. 4 shows how intravenous administration of Compound C significantly increased burst frequency of the diaphragm in anesthetized rats. Effects of Compound C on the burst frequency of the diaphragm. Mean values in burst frequency during 2 min before vehicle or Compound C administration were used as control values, and those during 10 min after vehicle or Compound C administration were used to calculate percent of control values. N=8. Data are presented as mean±SEM. *P<0.05, ***P<0.001 compared to vehicle-treated rats.

FIG. 5 shows how intravenous administration of Compound C significantly increased burst amplitude of the genioglossus muscle in anesthetized rats. Effects of Compound C on the burst amplitude of the genioglossus muscle. Mean values in burst amplitude during 2 min before vehicle or Compound C administration were used as control values, and those during 10 min after vehicle or Compound C administration were used to calculate percent of control values. N=8. Data are presented as mean±SEM. ***P<0.001 compared to vehicle-treated rats.

### DETAILED DESCRIPTION

The methods, compositions, and uses disclosed herein include the use of an OX2R agonist to improve respiratory function during sleep in a subject in need thereof. The methods, compositions, and uses disclosed herein also include the use of an OX2R agonist at non-awakening plasma concentration thereof. The orexin type 2 receptor agonist can be administered in an amount that is effective to improve respiratory function during sleep while not providing an arousal response or wakefulness in the subject.

The methods, compositions, and uses disclosed herein also include the use of an orexin 1 receptor (OX1R)/OX2R dual agonist, for example, the use of an OX1R/OX2R dual agonist to improve respiratory function during sleep in a subject in need thereof.

Generally, when a pharmacological substance is administered to a subject, the blood plasma concentration of the substance increases for a time, reaches a maximum concentration and then decays. Consequently, the medicinal effect of the substance generally follows its blood plasma concentration. In the case of an OX2R agonist administered to promote arousal in subjects (e.g., in subjects having Narcolepsy Type 1 (NT1)), once the blood plasma concentration of the agonist exceeds the maximum non-awakening concentration, the subjects experience marginal arousal, which is followed by full arousal once the concentration exceeds a certain threshold (the arousal-promoting concentration). As such, above this concentration, the OX2R agonist provides an arousal response or wakefulness in the subject. Certain OX2R agonists have been shown to produce potent efficacy in wakefulness when the blood plasma concentration of the OX2R agonist exceeds the arousal-promoting concentration (e.g., in subjects having NT1).

The present disclosure relates to the use of an OX2R agonist to improve respiratory function during sleep in a subject in need thereof. The present disclosure also relates to the use of an OX2R agonist at a non-awakening concentration that does not provide an arousal response or wakefulness in a subject (e.g. mammal). The present disclosure further relates to a method of administering an OX2R agonist to a subject (e.g. mammal) in need thereof at a dose that provides a blood plasma concentration of the agonist after administration at or below the maximum non-awakening blood plasma concentration thereof. The present disclosure also relates to a method of regulating inspiratory air flow in a subject by administering an OX2R agonist. The present disclosure also relates to a method of improving respiratory function during sleep in a subject, wherein the orexin type 2 receptor agonist is administered in an amount that is effective to improve respiratory function during sleep while not providing an arousal response or wakefulness in the subject.

The present disclosure also relates to a method of treating sleep apnea in a subject (e.g. mammal) in need thereof by maintaining a non-zero blood plasma concentration of an OX2R agonist after administration at or below the maximum non-awakening concentration of the agonist over multiple dosing intervals (i.e., chronically) in the subject. In some embodiments, the blood plasma concentration of an OX2R agonist after administration is about 1/20 to about 1/1 of the maximum non-awakening concentration of the agonist over the dosing interval.

The present disclosure also relates to a dosing regimen for improving respiratory function during sleep in a subject (e.g. mammal) by administering an OX2R agonist at a dose sufficient to provide a plasma blood concentration of the OX2R agonist at or below a maximum non-awakening plasma concentration of the agonist over a dosing interval.

The dosing regimen may also be sufficient to provide a blood plasma concentration of the agonist that is equivalent to a blood plasma concentration of from about 5 ng/ml to about 100 ng/ml of methyl 3-((methylsulfonyl)amino)-2-(((4-phenylcyclohexyl)oxy)methyl) piperidine-1-carboxylate over a dosing interval after administering to the subject the methyl 3-((methylsulfonyl)amino)-2-(((4-phenylcyclohexyl)oxy)methyl) piperidine-1-carboxylate, or a pharmaceutically acceptable salt thereof. The dosing regimen may further be sufficient to provide a blood plasma concentration of the methyl 3-((methylsulfonyl)amino)-2-(((4-phenylcyclohexyl)oxy)methyl) piperidine-1-carboxylate is about 10 ng/ml or about 30 ng/mL.

The present disclosure focuses on the effect of OX2R agonists for improving respiratory function during sleep at a non-awakening blood plasma concentration, i.e., an amount that is effective to improve respiratory function while not providing an arousal response or wakefulness in the subject. Administration of OX2R agonists at a non-awakening blood plasma concentration unexpectedly improves respiratory function during sleep and regulates inspiratory airflow in the subject. The use of OX2R agonists of the present disclosure is expected to provide a new method and strategy for improving respiratory function during sleep and for treating sleep apnea, including obstructive sleep apnea (OSA), treatment emergent sleep apnea, central sleep apnea (CSA), and mixed sleep apnea. Further, the use of OX2R agonists of the present disclosure is expected to have fewer side effects as compared to the use of the agonists in arousal-promoting concentrations.

In one aspect, the disclosure provides a method for improving respiratory function during sleep in a subject in need thereof, comprising administering to the subject an OX2R agonist. In some embodiments, the administration of the OX2R agonist regulates inspiratory air flow in the subject. In some embodiments, the administration of the OX2R agonist regulates respiratory rhythm in the subject. In some embodiments, the administration of the OX2R agonist activates the hypoglossal motoneurons. In some embodiments, the administration of the OX2R agonist regulates activity of the genioglossus muscle. In some embodiments, the administration of the OX2R agonist activates neurons in the pre-Bötzinger complex. In some embodiments, the administration of the OX2R agonist activates phrenic motoneurons. In some embodiments, the administration of the OX2R agonist regulates activity of the diaphragm.

In some embodiments, the subject is a mammal. In some embodiments, the subject is a human. In some embodiments, the subject has not been diagnosed with Narcolepsy Type 1 (NT1). In some embodiments, the subject has not had surgery requiring general anesthesia up to 5 years, up to 4 years, up to 3 years, up to 2 years, or up to 1 year prior to administration of an OX2R agonist as disclosed herein. In some embodiments, the subject has not experienced sedation and respiratory depression due to opioids.

In one aspect, the disclosure provides a method for regulating inspiratory airflow in the subject, comprising administering to the subject an OX2R agonist. In another aspect, the disclosure provides a method for regulating respiratory rhythm in the subject, comprising administering to the subject an OX2R agonist.

In some embodiments, the subject has sleep apnea. In some embodiments, the subject has OSA. In some embodiments, the subject has CSA. In some embodiments, the subject has mixed sleep apnea. In some embodiments, the subject has treatment emergent sleep apnea.

In some embodiments, the OX2R agonist is a OX1R/OX2R dual agonist. In some embodiments, the OX2R agonist is administered as a dose and/or over a dosing interval.

In some embodiments, the OX2R agonist is administered in an amount that is effective to improve respiratory function during sleep while not providing an arousal response or wakefulness in the subject. In some embodiments, administration of the OX2R agonist provides a blood plasma concentration of the agonist at or below a maximum non-awakening plasma concentration of the agonist. In some embodiments, administration of the OX2R agonist provides a blood plasma concentration of the agonist at or below a maximum non-awakening plasma concentration of the agonist over a dosing interval.

In some embodiments, the arousal response or wakefulness is determined by measuring sleep latency in one or more subjects diagnosed with sleep apnea at their usual bedtime.

In some embodiments, the blood plasma concentration of the OX2R agonist is selected by: i) determining the non-awakening plasma concentration of the OX2R agonist that does not provide an arousal response or wakefulness in a human; and ii) determining a dose of the OX2R agonist that will provide a blood plasma concentration which is at or below the maximum non-awakening plasma concentration.

In some embodiments, administration of the OX2R agonist reduces the Apnea Hypopnea Index (AHI) in the subject. In some embodiments, the methods disclosed herein provide an Apnea Hypopnea Index (AHI) in the subject that is reduced relative to the absence of administering the orexin type 2 receptor agonist. In some embodiments, the reduction in AHI is about 1 to about 50 events per hour. In some embodiments, the reduction in AHI is about 1 to about 40 events per hour. In some embodiments, the reduction in AHI is about 1 to about 30 events per hour. In some embodiments, the reduction in AHI is about 1 to about 20 events per hour. In some embodiments, the reduction in AHI is about 5 to about 50 events per hour. In some embodiments, the reduction in AHI is about 5 to about 40 events per hour. In some embodiments, the reduction in AHI is about 5 to about 30 events per hour. In some embodiments, the reduction in AHI is about 5 to about 20 events per hour. In some embodiments, the reduction in AHI is about 10 to about 50 events per hour. In some embodiments, the reduction in AHI is about 10 to about 40 events per hour. In some embodiments, the reduction in AHI is about 10 to about 20 events per hour.

In some embodiments, the reduction in AHI is about 1 event per hour, or about 2 events per hour, or about 3 events per hour, or about 4 events per hour, or about 5 events per hour, or about 6 events per hour, or about 7 events per hour, or about 8 events per hour, or about 9 events per hour, or about 10 events per hour, or about 11 events per hour, or about 12 events per hour, or about 13 events per hour, or about 14 events per hour, or about 15 events per hour, or about 16 events per hour, or about 17 events per hour, or about 18 events per hour, or about 19 events per hour, or about 20 events per hour, or about 22 events per hour, or about 24 events per hour, or about 26 events per hour, or about 28 events per hour, or about 30 events per hour, or about 35 events per hour, or about 40 events per hour, or about 45 events per hour, or about 50 events per hour. In some embodiments, the reduction in AHI is about 9 events per hour.

In some embodiments, administration of the OX2R agonist reduces the hypoxic burden in the subject. In some embodiments, the methods disclosed herein provide an hypoxic burden in the subject that is reduced relative to the absence of administering the orexin type 2 receptor agonist. In some embodiments, the hypoxic burden reduced to from about 45%-75%*minutes/hour. In some embodiments, the hypoxic burden reduced to from about 50%-70%*minutes/hour. In some embodiments, the hypoxic burden reduced to from about 55%-65%*minutes/hour. In some embodiments, the hypoxic burden reduced to about 60%*minutes/hour.

In some embodiments, administration of the OX2R agonist provides a plasma concentration of the agonist after administration of about 1/50 to about 1/1 of the maximum non-awakening concentration of the agonist. In some embodiments, the plasma concentration of the agonist after administration is about 1/40 to about 1/1 of the maximum non-awakening concentration of the agonist. In some embodiments, the plasma concentration of the agonist after administration is about 1/30 to about 1/1 of the maximum non-awakening concentration of the agonist. In some embodiments, the plasma concentration of the agonist after administration is about 1/20 to about 1/1 of the maximum non-awakening concentration of the agonist. In some embodiments, the plasma concentration of the agonist after administration is about 1/10 to about 1/1 of the maximum non-awakening concentration of the agonist. In some embodiments, the plasma concentration of the agonist after administration is about ⅕ to about 1/1 of the maximum non-awakening concentration of the agonist.

In some embodiments, administration of the OX2R agonist provides a blood plasma concentration of the agonist that is bioequivalent to a blood plasma concentration of from about 5 ng/ml to about 100 ng/mL, or any value in between, of methyl 3-((methylsulfonyl)amino)-2-(((4-phenylcyclohexyl)oxy)methyl) piperidine-1-carboxylate over a dosing interval after administering to the subject the methyl 3-((methylsulfonyl)amino)-2-(((4-phenylcyclohexyl)oxy)methyl) piperidine-1-carboxylate, or a pharmaceutically acceptable salt thereof. In some embodiments, the blood plasma concentration of the methyl 3-((methylsulfonyl)amino)-2-(((4-phenylcyclohexyl)oxy)methyl) piperidine-1-carboxylate is about 10 ng/ml to about 80 ng/ml. In some embodiments, the blood plasma concentration of the methyl 3-((methylsulfonyl)amino)-2-(((4-phenylcyclohexyl)oxy)methyl) piperidine-1-carboxylate is about 10 ng/ml to about 60 ng/mL. In some embodiments, the blood plasma concentration of the methyl 3-((methylsulfonyl)amino)-2-(((4-phenylcyclohexyl)oxy)methyl) piperidine-1-carboxylate is about 10 ng/mL to about 50 ng/mL. In some embodiments, the blood plasma concentration of the methyl 3-((methylsulfonyl)amino)-2-(((4-phenylcyclohexyl)oxy)methyl) piperidine-1-carboxylate is about 10 ng/ml to about 40 ng/ml. In some embodiments, the blood plasma concentration of the methyl 3-((methylsulfonyl)amino)-2-(((4-phenylcyclohexyl)oxy)methyl) piperidine-1-carboxylate is about 10 ng/ml to about 30 ng/ml. In some embodiments, the blood plasma concentration of the methyl 3-((methylsulfonyl)amino)-2-(((4-phenylcyclohexyl)oxy)methyl) piperidine-1-carboxylate is about 5 ng/mL. In some embodiments, the blood plasma concentration of the methyl 3-((methylsulfonyl)amino)-2-(((4-phenylcyclohexyl)oxy)methyl) piperidine-1-carboxylate is about 10 ng/mL. In some embodiments, the blood plasma concentration of the methyl 3-((methylsulfonyl)amino)-2-(((4-phenylcyclohexyl)oxy)methyl) piperidine-1-carboxylate is about 20 ng/mL. In some embodiments, the blood plasma concentration of the methyl 3-((methylsulfonyl)amino)-2-(((4-phenylcyclohexyl)oxy)methyl) piperidine-1-carboxylate is about 30 ng/mL. In some embodiments, the blood plasma concentration of the methyl 3-((methylsulfonyl)amino)-2-(((4-phenylcyclohexyl)oxy)methyl) piperidine-1-carboxylate is about 40 ng/mL. In some embodiments, the blood plasma concentration of the methyl 3-((methylsulfonyl)amino)-2-(((4-phenylcyclohexyl)oxy)methyl) piperidine-1-carboxylate is about 50 ng/mL. In some embodiments, the blood plasma concentration of the methyl 3-((methylsulfonyl)amino)-2-(((4-phenylcyclohexyl)oxy)methyl) piperidine-1-carboxylate is about 60 ng/mL. In some embodiments, the blood plasma concentration of the methyl 3-((methylsulfonyl)amino)-2-(((4-phenylcyclohexyl)oxy)methyl) piperidine-1-carboxylate is about 70 ng/mL. In some embodiments, the blood plasma concentration of the methyl 3-((methylsulfonyl)amino)-2-(((4-phenylcyclohexyl)oxy)methyl) piperidine-1-carboxylate is about 80 ng/mL. In some embodiments, the blood plasma concentration of the methyl 3-((methylsulfonyl)amino)-2-(((4-phenylcyclohexyl)oxy)methyl) piperidine-1-carboxylate is about 90 ng/mL. In some embodiments, the blood plasma concentration of the methyl 3-((methylsulfonyl)amino)-2-(((4-phenylcyclohexyl)oxy)methyl) piperidine-1-carboxylate is about 100 ng/mL.

In some embodiments, the OX2R agonist can be administered using any method known to a person of skill in the art. In some embodiments, the OX2R agonist is administered orally, intravenously, subcutaneously, transdermally, or transmucosally. In some embodiments, the OX2R agonist is administered orally. In some embodiments, the OX2R agonist is administered intravenously. In some embodiments, the OX2R agonist is administered subcutaneously. In some embodiments, the OX2R agonist is administered transdermally. In some embodiments, the OX2R agonist is administered transmucosally.

It will be understood that a suitable dose of the OX2R agonist, and optionally one or more additional therapeutic agents, may be taken at any time of the day or night. In some embodiments, the OX2R agonist is administered in the morning. In some embodiments, the OX2R agonist is administered in the middle of the day, including, for example, at about 12:00 μm or the subject's lunch time. In some embodiments, the OX2R agonist is administered in the evening. In some embodiments, the OX2R agonist is administered in the morning and the evening. It will be understood that a suitable dose of the OX2R agonist, and optionally one or more additional therapeutic agents, may be administered with food or without food (i.e., fasting). In some embodiments, the OX2R agonist is administered with food, or with a meal. In some embodiments, the OX2R agonist is administered while fasting.

In some embodiments, the OX2R agonist is administered on a daily schedule. In some embodiments, the OX2R agonist is administered every other day. In some embodiments, the OX2R agonist is administered once every three days. In some embodiments, the OX2R agonist is administered on a twice-weekly schedule. In some embodiments, the OX2R agonist is administered on a three times a week schedule. In some embodiments, the OX2R agonist is administered on a weekly schedule. In some embodiments, the OX2R agonist is administered on a once every two weeks schedule. In some embodiments, the OX2R agonist is administered on a once every three weeks schedule. In some embodiments, the OX2R agonist is administered on a once every four weeks schedule. In some embodiments, the OX2R agonist is administered on a once every month schedule.

In some embodiments, the OX2R agonist is administered once per day. In some embodiments, the OX2R agonist is administered twice per day. In some embodiments, the OX2R agonist is administered three times per day. In some embodiments, the OX2R agonist is administered four times per day.

In some embodiments, the OX2R agonist is administered prior to the subject's bedtime. In some embodiments, the OX2R agonist is administered from about 5 minutes to about 5 hours before the subject's bedtime, or any value of time in between. In some embodiments, the OX2R agonist is administered from about 5 minutes to about 4 hours before the subject's bedtime. In some embodiments, the OX2R agonist is administered from about 5 minutes to about 3 hours before the subject's bedtime. In some embodiments, the OX2R agonist is administered from about 5 minutes to about 1 hours before the subject's bedtime. In some embodiments, the OX2R agonist is administered from about 5 minutes to about 1 hour before the subject's bedtime. In some embodiments, the OX2R agonist is administered from about 5 minutes to about 45 minutes before the subject's bedtime. In some embodiments, the OX2R agonist is administered from about 5 minutes to about 30 minutes before the subject's bedtime. In some embodiments, the OX2R agonist is administered from about 5 minutes to about 20 minutes before the subject's bedtime. In some embodiments, the OX2R agonist is administered at about 5 minutes before bedtime, or about 10 minutes before bedtime, or about 20 minutes before bedtime, or about 30 minutes before bedtime, or about 45 minutes before bedtime, or about 1 hour before bedtime, or about 2 hours before bedtime, or about 3 hours before bedtime, or about 4 hours before bedtime, or about 5 hours before bedtime.

In some embodiments, the OX2R agonist is administered in an amount of from about 0.01 mg to about 1.0 g, or any value in between. In some embodiments, the OX2R agonist is administered in an amount of from about 0.1 mg to about 500 mg. In some embodiments, the OX2R agonist is administered in an amount of from about 1 mg to about 100 mg. In some embodiments, the OX2R agonist is administered in an amount of from about 1 mg to about 50 mg. In some embodiments, the OX2R agonist is administered in an amount of from about 1 mg to about 20 mg. In some embodiments, the OX2R agonist is administered in an amount of from about 1 mg to about 10 mg. In some embodiments, the OX2R agonist is administered in an amount of from about 1 mg to about 5 mg. In some embodiments, the OX2R agonist is administered in an amount of about 0.01 mg. In some embodiments, the OX2R agonist is administered in an amount of about 0.1 mg. In some embodiments, the OX2R agonist is administered in an amount of about 1 mg. In some embodiments, the OX2R agonist is administered in an amount of about 2 mg. In some embodiments, the OX2R agonist is administered in an amount of about 3 mg. In some embodiments, the OX2R agonist is administered in an amount of about 4 mg. In some embodiments, the OX2R agonist is administered in an amount of about 5 mg. In some embodiments, the OX2R agonist is administered in an amount of about 10 mg. In some embodiments, the OX2R agonist is administered in an amount of about 20 mg. In some embodiments, the OX2R agonist is administered in an amount of about 50 mg. In some embodiments, the OX2R agonist is administered in an amount of about 100 mg. In some embodiments, the OX2R agonist is administered in an amount of about 200 mg. In some embodiments, the OX2R agonist is administered in an amount of about 300 mg. In some embodiments, the OX2R agonist is administered in an amount of about 400 mg. In some embodiments, the OX2R agonist is administered in an amount of about 500 mg. In some embodiments, the OX2R agonist is administered in an amount of about 1.0 g.

In one embodiment, the methods, compositions and uses of this disclosure may be directed to [1] the use of an orexin type 2 receptor agonist in a subject (e.g. mammal) at a blood plasma concentration which is at or below the maximum non-awakening plasma concentration of the OX2R agonist.

In another embodiment, the methods, compositions and uses of this disclosure may be directed to [2] the use of an orexin type 2 receptor agonist in a subject (e.g. mammal) at a dose that provides a blood plasma concentration which is at or below the maximum non-awakening plasma concentration of the OX2R agonist.

In another embodiment, the methods, compositions and uses of this disclosure may be directed to [3] a method comprising the administration of an orexin type 2 receptor agonist to a subject (e.g. mammal) at a blood plasma concentration which is at or below the maximum non-awakening plasma concentration of the OX2R agonist.

In another embodiment, the methods, compositions and uses of this disclosure may be directed to [4] a method of administration of an orexin type 2 receptor agonist to a subject (e.g. mammal) at a dose which provides a blood plasma concentration that is at or below the maximum non-awakening plasma concentration of the OX2R agonist.

In another embodiment, the methods, compositions and uses of this disclosure may be directed to [5] a method of treating sleep apnea in a subject (e.g. mammal), wherein the method comprises administering to the subject an orexin type 2 receptor agonist at a dose that provides a blood plasma concentration after administration which is at or below the maximum non-awakening plasma concentration of the OX2R agonist.

In another embodiment, the methods, compositions and uses of this disclosure may be directed to [6] a method of treating obstructive sleep apnea, central sleep apnea, mixed sleep apnea, or treatment emergent sleep apnea in a subject (e.g. mammal), wherein the method comprises administering to the subject an orexin type 2 receptor agonist at a dose that provides a blood plasma concentration after administration which is at or below the maximum non-awakening plasma concentration of the OX2R agonist.

In one embodiment, the methods, compositions and uses of this disclosure may be directed to [7] the use of an orexin type 2 receptor agonist in a subject (e.g. mammal) at a blood plasma concentration which is at or below the maximum non-awakening plasma concentration of the OX2R agonist for the treatment of sleep apnea in the subject.

In one embodiment, the methods, compositions and uses of this disclosure may be directed to [8] the use of an orexin type 2 receptor agonist in a subject (e.g. mammal) at a blood plasma concentration which is at or below the maximum non-awakening plasma concentration of the OX2R agonist for the treatment of obstructive sleep apnea, central sleep apnea, mixed sleep apnea, or treatment emergent sleep apnea during sleep phase in the subject.

In one embodiment, the methods, compositions and uses of this disclosure may be directed to [9] a method of administration of an orexin type 2 receptor agonist to a subject (e.g. mammal) at a dose that provides a blood plasma concentration after administration which is at or below the maximum non-awakening plasma concentration of the OX2R agonist for the treatment of sleep apnea in the subject.

In one embodiment, the methods, compositions and uses of this disclosure may be directed to [10] a method of administration of an orexin type 2 receptor agonist to a subject (e.g. mammal) at a dose that provides a blood plasma concentration after administration which is at or below the maximum non-awakening plasma concentration of the agonist for the treatment of obstructive sleep apnea, central sleep apnea, mixed sleep apnea, or treatment emergent sleep apnea during sleep phase in the subject.

In one embodiment, the methods, compositions and uses of this disclosure may be directed to [11] the method of administration of [9] or [10], wherein the orexin type 2 receptor agonist is administered in a sustained-release formulation.

In one embodiment, the methods, compositions and uses of this disclosure may be directed to [12] the method of administration of [9] or [10], wherein the orexin type 2 receptor agonist is administered in an oral formulation.

In an embodiment, the methods, compositions and uses of this disclosure may be directed to [13] repeated or continuous use of an orexin type 2 receptor agonist in a subject (e.g. mammal) at a blood plasma concentration which is at or below the maximum non-awakening plasma concentration of the OX2R agonist for the treatment of sleep apnea in the subject.

In another embodiment, the methods, compositions and uses of this disclosure may be directed to [14] repeated or continuous use of an orexin type 2 receptor agonist in a subject (e.g. mammal) at a blood plasma concentration which is at or below the maximum non-awakening plasma concentration of the OX2R agonist for the treatment of obstructive sleep apnea, central sleep apnea, mixed sleep apnea, or treatment emergent sleep apnea during sleep phase in the subject.

In an embodiment, the methods, compositions and uses of this disclosure may be directed to [15] a method of treating sleep apnea in a human in need thereof, the method comprising: administering to the human a dosage form comprising an orexin type 2 receptor agonist, wherein the dosage form provides a blood plasma concentration of the OX2R agonist after administration which is at or below the maximum non-awakening plasma concentration of the agonist over a dosing interval.

In one embodiment, [16] the method according to [15], wherein the blood plasma concentration of the orexin type 2 receptor agonist after administration reaches and maintains between about 5 percent to about 100 percent, about 10 percent to about 100 percent, about 15 percent to about 100 percent, about 20 percent to about 100 percent, about 30 percent to about 100 percent, or about 50 percent to about 100 percent of the maximum non-awakening plasma concentration of the agonist over a dosing interval.

In one embodiment, [17] the method according to [15], wherein the blood plasma concentration of the orexin type 2 receptor agonist after administration reaches and maintains between about 33 percent to about 100 percent of the maximum non-awakening plasma concentration of the agonist over a dosing interval.

In one embodiment, [18] the method according to any one of embodiments [15] to [17], wherein the dosing interval for the orexin type 2 receptor agonist is one day (once daily), two days (once every other day), a week (once per week), two weeks (once every two weeks), four weeks (once every four weeks), six weeks (once every six weeks), or eight weeks (once every eight weeks).

In one embodiment, [19] the method according to [18], wherein the blood plasma concentration of the orexin type 2 receptor agonist after administration is maintained for at least two weeks, at least four weeks, at least six weeks or at least eight weeks.

In one embodiment, [20] the method according to [15], wherein the blood plasma concentration of the orexin type 2 receptor agonist after administration reaches about one-fifth, one-quarter, one-third, or a half of the maximum non-awakening plasma concentration of the orexin type 2 receptor agonist.

In one embodiment, [21] the method according to [15], wherein the plasma concentration of the orexin type 2 receptor agonist after administration reaches the maximum non-awakening concentration thereof.

In one embodiment, [22] the method according to any one of embodiments [15] to [21], wherein the method improves one or more nighttime symptoms associated with sleep apnea.

In one embodiment, [23] the method according to any one of embodiments [15] to [21], wherein the method improves one or more daytime symptoms associated with sleep apnea.

In one embodiment, [24] the method according to any one of embodiments [15] to [21], wherein the method improves one or more daytime symptoms and one or more nighttime symptoms in patients with sleep apnea.

In one embodiment, [25] the method according to any one of embodiments [15] to [21], wherein the method improves one or more daytime symptoms and one or more nighttime symptoms in patients with obstructive sleep apnea, central sleep apnea, mixed sleep apnea, or treatment emergent sleep apnea.

In one embodiment, [26] the method according to any one of embodiments [15] to [25], wherein the orexin type 2 receptor agonist is administered to the human in a sustained release formulation.

In one embodiment, [27] the method according to [26], wherein the sustained release formulation is a depot formulation for subcutaneous administration.

In one embodiment, [28] the method according to [26], wherein the orexin type 2 receptor agonist is administered by an infusion system designed to provide continuous sub-cutaneous delivery of the OX2R agonist to the human.

In one embodiment, [29] the method according to [26], wherein the orexin type 2 receptor agonist is a compound having short half-life.

In one embodiment, [30] the method according to any one of embodiments [15] to [29], wherein the orexin type 2 receptor agonist is selected from N-((2S,3S)-1-(2-hydroxy-2-methylpropanoyl)-2-((2,3′,5′-trifluorobiphenyl-3-yl)methyl) pyrrolidine-3-yl) methanesulfonamide; N-((2S,3S)-2-((2,3′-difluorobiphenyl-3-yl)methyl)-1-(2-hydroxy-2-methylpropanoyl) pyrrolidine-3-yl) ethanesulfonamide; methyl(2R,3S)-3-((methylsulfonyl)amino)-2-(((cis-4-phenylcyclohexyl)oxy)methyl) piperidine-1-carboxylate; N-{(2S,3R)-4,4-difluoro-1-(2-hydroxy-2-methylpropanoyl)-2-[(2,3′,5′-trifluoro[1,1′-biphenyl]-3-yl)methyl]pyrrolidine-3-yl}methanesulfonamide; 4-(5-cyclopropyl-1,2,4-oxadiazol-3-yl)-N-{(1R,6S)-2,2-difluoro-6-[4-(propan-2-yl) piperazin- 1-yl]cyclohexyl}-4-methylpiperidine-1-carboxamide; N-{(1R,6S)-2,2-difluoro-6-[4-(propan-2-yl) piperazin-1-yl]cyclohexyl}-4-{5-[(1S,2S)-2-fluorocyclopropyl]-1,2,4-oxadiazol-3-yl}-4-methylpiperidine-1-carboxamide; (2R)-2-cyclopropyl-2-{(1R,3S,5S)-3-[(3S,4R)-1-(5-fluoropyrimidin-2-yl)-3-methoxypiperidin-4-yl]-8-azabicyclo[3.2.1]octan-8-yl}acetamide; I-2-((1R,3S,5S)-3-((3S,4R)-1-(5-fluoropyrimidin-2-yl)-3-methoxypiperidin-4-yl)-8-azabicyclo[3.2.1]octan-8-yl)-3-methylbutaneamidI(R)-2-((1R,3S,5S)-3-((3S,4R)-1-(5-chloropyrimidin-2-yl)-3-ethoxypiperidin-4-yl)-8-azabicyclo[3.2.1]octan-8-yl)-2-cyclopropyl acetamide; (R)-2-cyclopropyl-2-((1R,3S,5S)-3-((2S,4S)-1-(5-fluoropyrimidin-2-yl)-2-methylpiperidin-4-yl)-8-azabicyclo[3.2.1]octan-8-yl) acetamide; and N-((2¹S,2⁴S,5²R,5³S)-6-oxo-3,8-dioxa-1 (2,3)-pyrazina-5 (2,1)-piperidina-2 (1,4)-cyclohexanacyclooctaphane-5³-yl) methanesulfonamide; or a pharmaceutically acceptable salt or hydrate thereof.

In one embodiment, [31] the method according to any one of embodiments [15] to [29], wherein the orexin type 2 receptor agonist is N-((2S,3S)-1-(2-hydroxy-2-methylpropanoyl)-2-((2,3′,5′-trifluorobiphenyl-3-yl)methyl) pyrrolidine-3-yl) methanesulfonamide, or a pharmaceutically acceptable salt or hydrate thereof.

In one embodiment, [32] the method according to any one of embodiments [15] to [29], wherein the orexin type 2 receptor agonist is methyl(2R,3S)-3-((methylsulfonyl)amino)-2-(((cis-4-phenylcyclohexyl)oxy)methyl) piperidine-1-carboxylate, or a pharmaceutically acceptable salt or hydrate thereof.

In one embodiment, [33] the method according to any one of embodiments [15] to [29], wherein the orexin type 2 receptor agonist is N-{(2S,3R)-4,4-difluoro-1-(2-hydroxy-2-methylpropanoyl)-2-[(2,3′,5′-trifluoro[1,1′-biphenyl]-3-yl)methyl) pyrrolidine-3-yl}methanesulfonamide, or a pharmaceutically acceptable salt or hydrate thereof.

In one embodiment, [34] the method according to any one of embodiments [15] to [29], wherein the orexin type 2 receptor agonist is selected from 4-(5-cyclopropyl-1,2,4-oxadiazol-3-yl)-N-{(1R,6S)-2,2-difluoro-6-[4-(propan-2-yl) piperazin-1-yl]cyclohexyl}-4-methylpiperidine-1-carboxamide and N-{(1R,6S)-2,2-difluoro-6-[4-(propan-2-yl) piperazin-1-yl]cyclohexyl}-4-{5-[(1S,2S)-2-fluorocyclopropyl]-1,2,4-oxadiazol-3-yl}-4-methylpiperidine-1-carboxamide; or a pharmaceutically acceptable salt or hydrate thereof.

In one embodiment, [35] the method according to any one of embodiments [15] to [29], wherein the orexin type 2 receptor agonist is selected from (2R)-2-cyclopropyl-2-{(1R,3S,5S)-3-[(3S,4R)-1-(5-fluoropyrimidin-2-yl)-3-methoxypiperidin-4-yl]-8-azabicyclo[3.2.1]octan-8-yl}acetamide; (R)-2-((1R,3S,5S)-3-((3S,4R)-1-(5-fluoropyrimidin-2-yl)-3-methoxypiperidin-4-yl)-8-azabicyclo[3.2.1]octan-8-yl)-3-methylbutaneamide; (R)-2-((1R,3S,5S)-3-((3S,4R)-1-(5-chloropyrimidin-2-yl)-3-ethoxypiperidin-4-yl)-8-azabicyclo[3.2.1]octan-8-yl)-2-cyclopropyl acetamide; and (R)-2-cyclopropyl-2-((1R,3S,5S)-3-((2S,4S)-1-(5-fluoropyrimidin-2-yl)-2-methylpiperidin-4-yl)-8-azabicyclo[3.2.1]octan-8-yl) acetamide; or a pharmaceutically acceptable salt or hydrate thereof.

In one embodiment, [36] the method according to any one of embodiments [15] to [29], wherein the orexin type 2 receptor agonist is N-((2¹S,2⁴S,5²R,5³S)-6-oxo-3,8-dioxa-1 (2,3)-pyrazina-5 (2,1)-piperidina-2 (1,4)-cyclohexanacyclooctaphane-5³-yl) methanesulfonamide, or a pharmaceutically acceptable salt or hydrate thereof.

In one embodiment, [37] the method according to any one of embodiments [15] to [36], wherein the blood plasma concentration of the orexin type 2 receptor agonist is selected by: i) determining a non-awakening plasma concentration of the orexin type 2 receptor agonist that does not provide an arousal response in a human; and ii) determining a dose of the orexin type 2 receptor agonist that will provide a blood plasma concentration which is at or below the maximum non-awakening plasma concentration of the agonist determined in i).

In one embodiment, [38] the method according to any one of embodiments [15] to [36], wherein the blood plasma concentration of the orexin type 2 receptor agonist is selected by: i) determining a maximum non-awakening concentration of the orexin type 2 receptor agonist that does not provide an arousal response in a human; ii) determining the corresponding dose of the OX2R agonist that provides maximum non-awakening concentration of the agonist as determined in i); and iii) selecting a dose of the OX2R agonist that will provide a blood plasma concentration of the agonist that is at or below the maximum non-awakening plasma concentration of the agonist.

In one embodiment, [39] the method according to embodiments [37] or [38], wherein the arousal response or wakefulness can be determined by measuring sleep latency in one or more humans diagnosed with sleep apnea.

In one embodiment, [40] the method according to any one of embodiments [15] to [36], where the blood plasma concentration of the orexin type 2 receptor agonist is selected from concentrations that are at or below the maximum blood plasma concentration of the orexin type 2 receptor agonist that does not provide an arousal response or wakefulness in a human.

In another embodiment, the methods, compositions and uses of this disclosure may be directed to [41] the use of an orexin type 2 receptor agonist in a human, at a blood plasma concentration that is at or below the maximum non-awakening plasma concentration of the agonist, in combination with an additional orexin type 2 receptor agonist at a blood plasma concentration that is at or below the maximum non-awakening plasma concentration of the additional OX2R agonist.

In one embodiment, the methods, compositions and uses of this disclosure may be directed to [42] a method of treatment of sleep apnea in a subject (e.g. mammal), the method comprising: i) acute treatment for sleep apnea which includes administering an orexin type 2 receptor agonist to the subject at a blood plasma concentration that is above the maximum non-awakening concentration of the OX2R agonist; ii) followed by maintenance treatment comprising repeatedly or continuously administering the OX2R agonist to the subject at a blood plasma concentration that is at or below the maximum non-awakening concentration of the OX2R agonist.

In another embodiment, the methods, compositions and uses of this disclosure may be directed to [43] an orexin type 2 receptor agonist which is selected from compounds described in any of WO 2019/027058, WO2017/135306, WO2020/158958, WO2021/107023, WO2022/014680, WO2020/167701, WO2020/167706, WO2021/026047, WO2022/040070, WO2022/094012, WO2022/040058, WO2022/109117, WO 2022/119888, WO 2022/132696, WO2022/051583, WO2022/051596, and WO2021/108628.

In another embodiment, the methods, compositions and uses of this disclosure may be directed to [44] a pharmaceutical composition which comprises an orexin type 2 receptor agonist at a dose which provides a blood plasma concentration of the agonist at or below the maximum non-awakening plasma concentration over a dosing interval.

In one embodiment, [45] the dose of orexin type 2 receptor agonist according to [44] provides a blood plasma concentration which is not less than about 1/20 of the maximum non-awakening plasma concentration.

In one embodiment, [46] the dose of orexin type 2 receptor agonist according to [44] provides a blood plasma concentration which is not less than about 1/10 of the maximum non-awakening plasma concentration.

In one embodiment, [47] the dose of orexin type 2 receptor agonist according to [44] provides a blood plasma concentration which is not less than about ⅕ of the maximum non-awakening plasma concentration.

In one embodiment, [48] the dose of orexin type 2 receptor agonist according to [44] provides a blood plasma concentration which is not less than about ⅓ of the maximum non-awakening plasma concentration.

In one embodiment, [49] the dose of orexin type 2 receptor agonist according to [44] provides a blood plasma concentration which is not less than about ½ of the maximum non-awakening plasma concentration.

In one embodiment, [50] the dose of orexin type 2 receptor agonist according to [44] provides a blood plasma concentration which is not less than about the maximum non-awakening plasma concentration.

In one embodiment, the methods, compositions and uses of this disclosure may be directed to [51] a dose of an orexin type 2 receptor agonist for administration to a human, wherein the dose is in a range of about 1 mg to about 100 mg, or about 1 mg to about 50 mg, or about 1 mg to about 10 mg, or about 1 mg to about 5 mg.

In another embodiment, the methods, compositions and uses of this disclosure may be directed to [52] a method of administering an orexin type 2 receptor agonist to a mammal in need thereof, wherein the method comprises maintaining the average blood plasma concentration of the agonist in the mammal which is less than about 100 ng/ml, less than about 50 ng/ml, less than about 30 ng/ml, less than about 10 ng/ml, less than about 5 ng/mL, less than about 3 ng/mL, or less than about Ing/mL, but greater than 0 ng/ml after administration or over the dosing interval.

Another aspect of the present disclosure relates to [53] a method of producing a dosage form for treatment of sleep apnea in a human in need thereof, which comprises an orexin type 2 receptor agonist in an amount of about 1 mg to about 5 mg, wherein the dosage form is an oral dosage form, or a sustained release formulation for injection.

Another aspect of the present disclosure relates to [54] the use of an orexin type 2 receptor agonist in manufacturing a dosage form for treatment of sleep apnea in a human in need thereof, wherein the agonist provides a blood plasma concentration after administration which is at or below the maximum non-awakening concentration of the agonist, and the dosage form is an oral dosage form or a sustained release formulation for injection.

In one embodiment [55] of the use according to [54], the amount of the orexin type 2 receptor agonist in the dosage form is about 1 mg to about 100 mg, or about 1 mg to about 50 mg, or about 1 mg to about 10 mg, or about 1 mg to about 5 mg.

In another embodiment, the methods, compositions and uses of this disclosure may be directed to [56] a pharmaceutical composition comprising an orexin type 2 receptor agonist at a dose which provides a blood concentration after administration at or below the maximum non-awakening plasma concentration in a subject (i.e. mammal)

In another embodiment, the methods, compositions and uses of this disclosure may be directed to [57] a method of treating sleep apnea in a human in need thereof by administering an orexin type 2 receptor agonist, which method comprises i) obtaining the maximum non-awakening plasma concentration of the agonist in the human; ii) selecting a dose of the agonist that provides blood plasma concentration at or below the maximum non-awakening plasma concentration in the human; and iii) administering the dose of the agonist selected in ii) once, repeatedly, or continuously to the human.

In another embodiment, the methods, compositions and uses of this disclosure may be directed to [58] a method of treating sleep apnea in a human in need thereof by administering an orexin type 2 receptor agonist, which method comprises i) obtaining the maximum non-awakening plasma concentration of the agonist in the human; ii) selecting a plasma concentration at or below the maximum non-awakening plasma concentration in the human; and iii) administering the agonist at a dose that provides the plasma concentration selected in ii) once, repeatedly, or continuously to the human.

In another embodiment, the methods, compositions and uses of this disclosure may be directed to [59] a method of producing a pharmaceutical composition for treatment of sleep apnea in a human in need thereof comprising an orexin type 2 receptor agonist, wherein the method comprises: i) determining the maximum non-awakening concentration of the orexin type 2 receptor agonist in human with sleep apnea; ii) selecting a dose of the agonist that provides blood plasma concentration at or below the maximum non-awakening plasma concentration in human with sleep apnea; iii) combining the unit dose of the agonist and pharmaceutically acceptable carriers to formulate the pharmaceutical composition.

### Methods and Uses

The methods and uses disclosed herein may treat sleep apnea in a subject in need thereof. The methods and uses disclosed herein may also treat symptoms of sleep apnea. In some embodiments, treating sleep apnea may comprise reducing or alleviating one or more symptoms of sleep apnea. The one or more symptoms of sleep apnea may be selected from snoring, collapsed airway, blocked airway, gasping for air during sleep, dry mouth, headache, insomnia, excessive daytime sleepiness (EDS), hypersomnia, irritability, reduced mental focus, fatigue, Cheyne-Stokes breathing, night sweats, sexual dysfunction, dizziness, nightmares, and restless sleep. In some embodiments, the one or more symptoms of sleep apnea is selected from excessive daytime sleepiness (EDS), snoring, collapsed airway, blocked airway, and gasping for air during sleep. The methods and uses disclosed herein may also treat comorbidities of sleep apnea, such as hypertension, atrial fibrillation, congestive heart failure, stroke, metabolic syndrome, diabetes, including type 2 diabetes, obesity, depression, gastroesophageal reflux disease (GERD), diabetes mellitus, hypercholesterolemia, asthma, and the like. Sleep apnea may be diagnosed by diagnostic criteria generally used in the field, e.g., the third edition of the International Classification of Sleep Disorders (ICSD-3). Additionally, nighttime sleep will be improved, for example, by reducing the number of arousal responses and waking incidents during the sleep cycle.

The methods and uses disclosed herein may treat sleep apnea and increase wakefulness outside the sleep cycle, and/or decrease and/or treat excessive sleepiness in a subject in need thereof during active phase. In some embodiments, excessive sleepiness as used herein is also known as excessive daytime sleepiness (EDS). The methods and uses disclosed herein may also decrease sleep fragmentation. In some embodiments, wakefulness, excessive sleepiness and/or sleep fragmentation is determined by known method such as using any one or more of electroencephalogram (EEG), electromyogram (EMG), Maintenance Wakefulness Test (MWT), polysomnography, and the like (Sleep, Vol. 45, Issue 8, zsac091, (2022)). MWT is a validated objective measure of the time taken for a subject to fall asleep under soporific conditions (Electroencephalogr. Clin. Neurophysiol., 53 (6): 658-661, (1982). MWT is quantified by EEG optionally combined with EMG. An electroencephalogram (EEG) is a test that detects electrical activity in the brain using small, metal discs or electrodes attached to the scalp. In some embodiments, an arousal response, wakefulness, and/or decrease of sleepiness is determined by using the multiple sleep latency test (MSLT) or the Oxford Sleep Resistance (OSLER) test. In some embodiments, the test is the Karolinska Sleepiness Scale (KSS), the Epworth Sleepiness Scale (ESS) or the Stanford Sleepiness Scale. In some embodiments, treating sleep apnea may comprise reducing or alleviating one or more symptoms of sleep apnea. The one or more symptoms of sleep apnea may be selected from the symptoms listed herein.

In some embodiments, the subject suffers from the diseases or disorders or symptoms associated with sleep apnea and excessive sleepiness. In some embodiments, the subject is a sleep-deprived subject, a subject with excessive sleepiness, a subject with disruptive regular sleep/wake cycle, or a subject with a need to decrease sleepiness.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the term “administration” of an agent to a subject includes any route of introducing or delivering the agent to a subject to perform its intended function. Administration can be carried out by any suitable oral route or non-oral route, including, but not limited to, intravenously, intramuscularly, intraperitoneally, subcutaneously, and other suitable routes as described herein. Administration includes self-administration and the administration by another. Administration of an OX2R agonist for treatment purpose in this disclosure can generally be once, long-term, continuous, chronic, and/or repetitive. “After administration” of an OX2R agonist in the present disclosure means a certain time period elapses from the administration of the OX2R agonist to a subject. Generally, this means from about 24 to about 48 hours after the initial administration.

As used herein, the term “dosage form” or “pharmaceutical composition” means a composition containing one or more drug molecules. Examples of the dosage form include oral preparations such as tablet (including sugar-coated tablet, film-coated tablet, sublingual tablet, orally disintegrating tablet, buccal tablet), capsule (including soft capsule, microcapsule), pill, granule, powder, troche, syrup, liquid, emulsion, suspension, aerosol, films (e.g., orally disintegrable films, oral mucosa-adhesive film) and the like, parenteral agents such as injection (e.g., subcutaneous injection, intravenous injection, intramuscular injection, intraperitoneal injection, drip infusion), external preparation (e.g., transdermal absorption type preparation, ointment, lotion, adhesive preparation), suppository (e.g., rectal suppository, vaginal suppository), pellet, nasal preparation, pulmonary preparation (inhalant), eye drop and the like. The compound and medicament of the present disclosure can be respectively safely administered orally or parenterally (e.g., intrarectal, intravenous, intraarterial, intramuscular, subcutaneous, intraorgan, intranasal, intradermal, instillation, intracerebral, intravaginal, intraperitoneal, intratumoral, proximal tumor administrations, and administration to the lesion). These preparations may be a release control preparation (e.g., sustained-release microcapsule) such as an immediate-release preparation, a sustained-release preparation, and the like.

As used herein, the term “effective amount” or “therapeutically effective amount” refers to a quantity of a compound sufficient to achieve a desired effect or a desired therapeutic effect. In the context of therapeutic applications, the amount of the compound administered to the subject may depend on the type and severity of the disease or symptom and on the characteristics of the individual, such as general health, age, sex, body weight and tolerance to drugs. The skilled artisan will be able to determine appropriate dosages depending on these and other factors.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” or “decrease” refers to alter negatively by at least about 5% including, but not limited to, alter negatively by about 5%, by about 10%, by about 25%, by about 30%, by about 50%, by about 75%, or by about 100%.

As used herein, the term “orexin receptor 2 agonist” or “OX2R agonist” refers to a drug or substance, such as a small molecule, that activates OX2R. OX2R is a G-protein coupled receptor (GPCR) which interacts with Gq class of heterotrimetic G proteins and β-arrestins. Orexin-A (OX-A) and Orexin-B (OX-B) peptides are known to increase calcium mobilization (Cell, Vol. 92, 573-585, (1998)) and promote recruitment of β-arrestins (J. Biol. Chem. Vol. 286, No. 19, 16726-16733, (2011)) in OX2R-expressed recombinant cells. OX2R agonists such as Compound A (COMPOUND A) and YNT-185 have also been shown to increase calcium mobilization and promote recruitment of β-arrestins (Pharmacol. Biochem. Behav., 187 (2019), 172794; Proc. Natl Acad. Sci. USA., 114 (22), 5731-5736, (2017)). Therefore, OX2R agonistic activity of drug candidates may be evaluated by calcium mobilization assays or β-arrestin recruitment assays using OX2R-expressed cells. In some embodiments, the OX2R agonist is in the form of a pharmaceutically acceptable salt.

As used herein, the term “arousal” (including “arousal” in “arousal response” “arousal-promoting concentration”) and “wakefulness” means the status of a subject is near complete wakefulness which is examined by known measures such as Maintenance of Wakefulness Test (MWT) and EEG/EMG recordings.

As used herein, “blood plasma concentration” (sometimes simplified as “plasma concentration”) refers to a concentration of a pharmaceutical substance in blood plasma derived from a subject blood collected at certain time point. “Blood plasma concentration after administration” in the present disclosure means the blood plasma concentration of the pharmaceutical substance at a time point following to administration of the substance, for example, about 24 hours or later following to administration of the substance. “Average blood plasma concentration” means average of blood plasma concentrations of different time points 24 hours or later following the administration of a pharmaceutical substance in a subject.

As used herein, the terms “non-awakening plasma concentration” or “non-awakening concentration” refer to a plasma concentration of an orexin type 2 receptor agonist that will not induce arousal (e.g., an arousal response) or wakefulness in a subject. Non-awakening plasma concentration can be identified by conducting a multiple-dose study. Such study may be conducted as part of preclinical and clinical PK/PD studies during drug development. Among non-awakening plasma concentrations, the “maximum non-awakening plasma concentration” is the highest concentration above which marginal arousal effect occurs. The phrase “at or below the maximum non-awakening plasma concentration” of an OX2R agonist means the plasma concentration of the OX2R agonist in a subject is larger than 0 (zero) and the same or less than the maximum non-awakening plasma concentration. In some embodiments, the plasma concentration of the OX2R agonist in a subject over a dosing interval is at or below the maximum non-awakening plasmatic concentration, but not less than about 1/20, about 1/12, about 1/10, about ⅛, about ⅕, or about ⅓ of the maximum non-awakening concentration of the agonist. The average plasma concentration of an OX2R agonist can be about 1/20, about 1/10, about ⅛, about ⅕, about ¼, about ⅓, about ½, or 1/1 of the maximum non-awakening concentration of the agonist, ±5%. In some embodiments, the average plasma concentration of the OX2R agonist, at or below the maximum non-awakening plasma concentration, is about 1/60, 1/40 1/36, 1/30, 1/20/or 1/10 of the arousal-promoting plasma concentration.

As used herein, the term “arousal-promoting concentration” of an OX2R agonist refers to a threshold blood plasma concentration of the agonist which provides nearly complete wakefulness effect (i.e. maintenance of wakefulness for more than 75% in 10 min-bins.) in a subject. Certain OX2R agonists have been shown to produce potent efficacy in wakefulness when the blood plasma concentration of the OX2R agonist exceeded its arousal-promoting concentration (e.g., in NT1 subjects). The arousal-promoting concentration of an OX2R agonist is significantly higher than the maximum non-awakening concentration of the OX2R agonist.

The determination of blood plasma concentration can be performed with measures known to the skilled person in the art, including high-performance liquid chromatography-tandem mass spectrometry. The level is typically expressed as ng of analyte/mL. A maximum non-awakening concentration and an arousal-promoting concentration of an OX2R agonist in a subject can be determined by combining time-series measurement of blood plasma concentration with recording of nighttime and/or daytime sleep and awake patterns in the subject by known methods including EEG/EMG recording and actigraphy. When detecting the maximum non-awakening concentration or arousal-promoting concentration of an OX2R agonist in a non-NT1 subject, the measurement is performed during night time when autogenic orexin-A level of the subject is generally low.

As used herein, the term “actigraphy” refers to methods using miniaturized computerized wristwatch-like devices to monitor and collect data generated by movements (also referred to as accelerometry). Most actigraphs contain an analogue system to detect movements. In some devices, a piezo-electric beam detects movement.

As used herein, the term “treatment”, “treating”, or “treat” includes improvement, reduction, alleviation, or amelioration of one or more symptoms associated with a disease. In one embodiment of the methods and uses disclosed herein may improve one or more nighttime symptoms of sleep apnea. Such improvements can be assessed using any one or more of Polysomnography (PSG) with respiratory airflow and effort leads, MWT, MSLT, EEG, or EMG, by comparing the conditions before and after administration of an OX2R agonist to the subject, or by comparing the conditions between subjects administered placebo and administered an OX2R agonist. In one embodiment, an OX2R agonist of the present disclosure improves sleep apnea symptoms.

As used herein, the term “dosing interval” refers to the time between administrations of an OX2R agonists to a subject. The dosing interval of an OX2R agonist may depend on the pharmacokinetic profiles of the OX2R agonist as well as the dosage form of the OX2R agonist in a manner known to the person skilled in the art. When the dosage form is an oral dosage form, the preferable dosing interval may be three times a day, twice per day, once per day, every other day, once a week, once every two weeks, once every four weeks, once every six weeks or once every eight weeks, among others. When the dosage form is a sustained-release formulation for subcutaneous injection, the preferable dosing interval may be once a week, once every two weeks, once every four weeks, once every six weeks or once every eight weeks, among others.

As used herein, the term “subject” refers to a mammal including human, bovine, horse, dog, cat, monkey, mouse, and rat, and preferably refers to a human. In some embodiments, the subject is a human. In some embodiments, the subject has not been diagnosed with Narcolepsy Type 1 (NT1). In some embodiments, the subject has not had surgery requiring general anesthesia up to 5 years, up to 4 years, up to 3 years, up to 2 years, or up to 1 year prior to administration of an OX2R agonist as disclosed herein. In some embodiments, the subject has not experienced sedation and respiratory depression due to opioids.

As used herein, the term “pharmaceutically acceptable” substances refer to those substances which are suitable for administration to subjects.

The methods, compositions, and uses in this disclosure are characterized in that the blood plasma concentration of an orexin type 2 receptor (OX2R) agonist in a subject is at or below the maximum non-awakening plasma concentration of the agonist. In one embodiment, when an OX2R agonist is administered to a subject in need thereof, the blood plasma concentration of the OX2R agonist in the subject is at or below the maximum non-awakening plasma concentration of the agonist when measured at 24 hours or later after administration. By using certain formulations, such as oral, infusion, and sustained-release formulations, an initial release of the OX2R agonist may continue for about 24 hours to about 48 hours following administration, and the plasma concentration of the OX2R agonist may rise above the maximum non-awakening concentration, but does not reach the arousal-promoting concentration. When the blood plasma concentration of the OX2R agonist is at or below the maximum non-awakening concentration, it is larger than 0 (zero) and is the same as, or less than, the maximum non-awakening plasma concentration. In some embodiments, the plasma concentration of the OX2R agonist in a subject is about 1/20 to 1/1, about 1/12 to 1/1, about 1/10 to 1/1, about ⅛ to 1/1, about ⅕ to 1/1, or about ⅓ to 1/1 of the maximum non-awakening concentration of the agonist.

In one embodiment, the OX2R agonist is administered over multiple dosing intervals in a subject for the treatment of sleep apnea and/or sleep apnea symptoms, and throughout each dosing interval, the plasma concentration of the agonist is at or below the maximum non-awakening plasma concentration of the agonist. As used in this disclosure in connection with the administration of an OX2R agonist, the terms “repeated,” “repetitive,” “repeatedly,” “continued,” “continuously,” “chronic,” “chronically,” or “long-term” means the OX2R agonist is administered over multiple dosing intervals.

In one embodiment, the OX2R agonist is administered to a subject by single oral administration, repeated oral administration, or by infusion administration, or by using a slow-release formulation or a sustained-release formulation of the OX2R agonist. The suitable formulation may be selected based on the characteristics of the OX2R agonist. A sustained-release or slow-release formulation may be particularly useful for administering an OX2R agonist having a short half-life (i.e., less than 8 hours, less than 7 hours, less than 6 hours, less than 5 hours, or less than 4 hours).

The dose of an OX2R agonist may be determined by i) identifying the maximum non-awakening concentration of the OX2R agonist in a subject (e.g. by using EEG), and then ii) identifying the dose of the OX2R agonist that achieves the maximum non-awakening concentration after administration. The person skilled in the art in the pharmaceutical industry can select a dose that is lower than the dose determined in the step ii). In some embodiments, the blood plasma concentration of an OX2R agonist that is at or below the maximum non-awakening plasma concentration of the agonist in a human is about 0.01 ng/ml to about 1 mg/mL, about 0.03 ng/ml to about 300 ng/ml, about 0.05 ng/mL to about 100 ng/ml, about 0.08 ng/ml to about 50 ng/mL, about 1 ng/ml to about 30 ng/mL, about 1 ng/mL to about 100 ng/ml, about 10 ng/ml, about 20 ng/ml, about 30 ng/mL, about 40 ng/ml, about 50 ng/ml, about 60 ng/ml, about 70 ng/ml, about 80 ng/mL, about 90 ng/mL, or about 100 ng/mL. In some embodiments, the dose of an OX2R agonist that provides a blood plasma concentration which is at or below the maximum non-awakening plasma concentration of the agonist in a human is about 0.1 mg to about 50 mg, about 0.5 mg to about 30 mg, or about 1 mg to about 20 mg.

As used herein, the term “bioequivalent” means two products or active agents, including, for example, two OX2R agonists, are equal in the rate and extent to which the active pharmaceutical ingredient (API) of the products becomes available at the site(s) of drug action.

Tests for Increased Wakefulness and/or Decreased Excessive Sleepiness

The methods and uses disclosed herein may treat sleep apnea in a subject in need thereof. In some embodiments, treating sleep apnea may comprise reducing or alleviating one or more symptoms of sleep apnea. The one or more symptoms of sleep apnea may include excessive daytime sleepiness (EDS). Additionally, nighttime sleep is expected to be improved, for example, by reducing the number of waking incidents during the sleep cycle.

The methods and uses disclosed herein may increase wakefulness and/or decrease excessive sleepiness during non-sleeping or daytime hours in a subject in need thereof. In some embodiments, wakefulness and/or decrease of excessive sleepiness is determined by electroencephalogram (EEG), electromyogram (EMG) and electrooculogram (EOG), together forming the basis of a polysomnogram (PSG). These tests can also be employed to determine a threshold concentration for potent arousal and a non-awakening concentration for a particular OX2R agonist. In some embodiments, wakefulness and/or decrease of sleepiness is determined by using the Maintenance Wakefulness Test (MWT) with EEG, optionally combined with EMG. An electroencephalogram (EEG) is a test that detects electrical activity in the brain, for example, by using small, metal discs or electrodes attached to the scalp. In some embodiments, wakefulness and/or decrease of sleepiness is determined by using the multiple sleep latency test (MSLT) or the Oxford Sleep Resistance (OSLER) test. In some embodiments, the test is the Karolinska Sleepiness Scale (KSS), the Epworth Sleepiness Scale (ESS) or the Stanford Sleepiness Scale.

In some embodiments, actigraphy (also referred to as accelerometry) can be employed to study the effects of an OX2R agonist on sleep and wake patterns. The term actigraphy refers to methods using miniaturized computerized wristwatch-like devices to monitor and collect data generated by movements. Most actigraphs contain an analogue system to detect movements. In some devices, a piezo-electric beam detects movement. See, Sadeh et al., Sleep Medicine Reviews 6 (2); 113-124 (2002).

The methods and uses disclosed herein may decrease excessive sleepiness during non-sleeping or daytime hours or improve Karolinska Sleepiness Scale (KSS) rating in a subject in need thereof. In some embodiments, the KSS rating is improved 1, 2, 3, 4, or 5 or more ratings. In some embodiments, the subject has a KSS rating of 1, 2, 3, 4, or 5 after treatment.

The methods and uses disclosed herein may comprise performing one or more tests to quantify a subject's sleepiness during non-sleeping or daytime hours. In some embodiments, the test is selected from the multiple sleep latency test (MSLT), maintenance of wakefulness test (MWT), and the Oxford Sleep Resistance (OSLER) test. In some embodiments, the test is MWT. In some embodiments, the test is the Karolinska Sleepiness Scale (KSS), the Epworth Sleepiness Scale (ESS), the Stanford Sleepiness Scale, Ullanlinna Narcolepsy Scale (UNS), Work Limitations Questionnaire (WLQ), SF-8 (subset of SF-36 questionnaire) or a combination of these tests.

### Modes of Administration

The methods and uses disclosed herein comprise administering an OX2R agonist to a subject in need thereof. In some embodiments, the OX2R agonist is administered orally. In some embodiments, the OX2R agonist is administered non-orally. In some embodiments, the non-oral administration is intravenous administration, subcutaneous administration, transdermal administration, intradermal administration or transmucosal administration. In some embodiments, the non-oral administration is intravenous administration. In some embodiments, the non-oral administration is subcutaneous administration.

Dosage forms and delivery devices for particular routes are more fully described below.

In some embodiments, the plasma concentration for an OX2R agonist represents an average plasma concentration for a group of treated subjects and the time period of 1 hour or more begins at any time point following administration. As long as the average plasma concentration for a group of treated subjects meets the condition, “about XX ng/ml or more for a period of about 1 hour or more,” the plasma concentration for an individually treated subject may deviate from the condition.

### Frequency of Administration

OX2R agonists of the present disclosure can be administered once or over multiple dosing intervals. In some embodiments, an OX2R agonist of the present disclosure may be administered three times per day, twice per day, once daily, every other day, once a week, twice a week, bi-weekly, monthly, or bi-monthly.

### Combination Therapy

The methods of treating sleep apnea of the present disclosure may further comprise combining the administration of an OX2R agonist at or below the maximum non-awakening plasma concentration with administration of an additional OX2R agonist, wherein the combined plasma concentrations for the two OX2R agonists is below the maximum non-awakening plasma concentration. The additional OX2R agonist and the OX2R agonist may be different or the same. In this treatment method, the additional OX2R agonist may be administered to a subject in need to provide acute treatment of sleep apnea symptoms, followed by administration of the OX2R agonist to provide maintenance treatment. Between the administrations of the two OX2R agonists, there may be a dosing interval of 1 to 5 days depending on the clearance profile of the additional OX2R agonist.

### OX2R Agonists

A useful OX2R agonist for the methods, uses, or compositions of the present disclosure is a chemical molecule (compound) having OX2R agonist activity. Such compound may be selected from among known compounds or from newly designed/synthesized compounds. A compound having shorter half-life (i.e. less than 8 hours, less than 7 hours, less than 6 hours, less than 5 hours, or less than 4 hours) is particularly useful when formulating it in a slow-release dosage form.

In some embodiments, the OX2R agonist is a OX1R/OX2R dual agonist.

In some embodiments, the OX2R agonist is a pharmaceutically acceptable salt.

In some embodiments, the OX2R agonist is a compound represented by the formula (I):

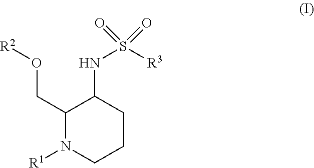

wherein
R¹ is
   (1) a hydrogen atom,
   (2) a C₁₋₆ alkyl-carbonyl group optionally substituted by 1 to 7 substituents selected from
      (i) a halogen atom, (ii) a cyano group, (iii) a hydroxy group, (iv) a C₃₋₁₀ cycloalkyl group, (v) a C₁₋₆ alkoxy group, (vi) a C₆₋₁₄ aryl group, (vii) a C₆₋₁₄ aryloxy group, (viii) a pyrazolyl group, a thiazolyl group, a pyrimidinyl group or a pyridazinyl group, each of which is optionally substituted by an oxo group, (ix) a pyrazolyloxy group optionally substituted by 1 to 3 C₁₋₆ alkyl groups, (x) a C₁₋₆ alkyl-carbonyl group, (xi) a C₁₋₆ alkoxy-carbonyl group, (xii) a C₁₋₆ alkyl-carbonyloxy group, (xiii) a C₁₋₆ alkylsulfonyl group, (xiv) a mono- or di-C₁₋₆ alkylamino group, (xv) a C₁₋₆ alkyl-carbonylamino group and (xvi) a (C₁₋₆ alkyl) (C₁₋₆ alkyl-carbonyl)amino group,
   (3) a C₃₋₁₀ cycloalkyl-carbonyl group optionally substituted by 1 to 3 substituents selected from a halogen atom, a cyano group, a hydroxy group, an oxo group and a C₁₋₆ alkyl group,
   (4) a C₁₋₆ alkoxy-carbonyl group optionally substituted by 1 to 6 substituents selected from deuterium, a halogen atom and a C₆₋₁₄ aryl group,
   (5) a C₃₋₁₀ cycloalkyloxy-carbonyl group optionally substituted by 1 to 3 substituents selected from a C₁₋₆ alkyl group,
   (6) a C₆₋₁₄ aryl-carbonyl group optionally substituted by 1 to 3 substituents selected from a halogen atom and a C₆₋₁₄ aryl group,
   (7) a C₆₋₁₄ aryloxy-carbonyl group,
   (8) a furylcarbonyl group, a thienylcarbonyl group, a pyrazolylcarbonyl group, an isoxazolylcarbonyl group or a pyridylcarbonyl group, each of which is optionally substituted by 1 to 3 substituents selected from a C₁₋₆ alkyl group,
   (9) an azetidinylcarbonyl group, an oxetanylcarbonyl group, a pyrrolidinylcarbonyl group, a tetrahydrofuranylcarbonyl group, a tetrahydropyranylcarbonyl group or a morpholinylcarbonyl group, each of which is optionally substituted by 1 to 3 substituents selected from an oxo group, a C₁-6 alkyl-carbonyl group, a C₁-6 alkoxy-carbonyl group and a C₁₋₆ alkylsulfonyl group,
   (10) a mono- or di-C₁₋₆ alkyl-carbamoyl group optionally substituted by 1 to 3 substituents selected from a halogen atom, a cyano group, a hydroxy group and a C₁₋₆ alkoxy group,
   (11) a mono- or di-C₃₋₁₀ cycloalkyl-carbamoyl group,
   (12) a mono- or di-C₆₋₁₄ aryl-carbamoyl group,
   (13) a C₁₋₆ alkylsulfonyl group,
   (14) a C₃₋₁₀ cycloalkylsulfonyl group,
   (15) a C₆₋₁₄ arylsulfonyl group optionally substituted by 1 to 3 halogen atoms,
   (16) a thienylsulfonyl group, a pyrazolylsulfonyl group, an imidazolylsulfonyl group, a pyridylsulfonyl group or a dihydrochromenylsulfonyl group, each of which is optionally substituted by 1 to 3 substituents selected from a C₁₋₆ alkyl group,
   (17) a mono- or di-C₁₋₆ alkyl-sulfamoyl group or
   (18) a C₁₋₆ alkyl-carbonyl-carbonyl group;
R² is a C₃₋₆ cycloalkyl group, a pyrrolidinyl group, a piperidinyl group or a dioxanyl group, each of which is optionally substituted by 1 to 3 substituents selected from
   (1) deuterium,
   (2) a halogen atom,
   (3) a hydroxy group,
   (4) a C₁₋₆ alkyl group optionally substituted by 1 to 3 substituents selected from a halogen atom and a C₆₋₁₄ aryl group,
   (5) a C₃₋₁₀ cycloalkyl group,
   (6) a C₁₋₆ alkoxy group optionally substituted by a C₃₋₁₀ cycloalkyl group,
   (7) a C₆₋₁₄ aryl group optionally substituted by 1 to 3 substituents selected from a halogen atom, a cyano group, a C₁-6 alkyl group optionally substituted by 1 to 3 halogen atoms, a C₁₋₆ alkoxy group optionally substituted by 1 to 3 halogen atoms and a hydroxy group,
   (8) a C₆₋₁₄ aryloxy group,
   (9) a tri-C₁₋₆ alkylsilyloxy group,
   (10) a pyrazolyl group, a thiazolyl group, a pyridyl group, a pyrimidinyl group, a quinazolinyl group, a benzothiazolyl group or an isoquinolinyl group, each of which is optionally substituted by 1 to 3 substituents selected from a halogen atom, a C₁₋₆ alkyl group and a C₁₋₆ alkoxy group, and
   (11) a C₆₋₁₄ aryl-carbonyl group; and
R³ is a C₁₋₆ alkyl group, or a mono- or di-C₁₋₆ alkylamino group,
or a pharmaceutically acceptable salt thereof.

In some embodiments, R¹ is
(1) a hydrogen atom,
(2) a C₁₋₆ alkyl-carbonyl group optionally substituted by a hydroxy group,
(3) a cyclopropanecarbonyl group,
(4) a C₁₋₆ alkoxy-carbonyl group or
(5) a mono- or di-C₁₋₆ alkyl-carbamoyl group;
R² is
(A) a cyclohexyl group optionally substituted by 1 to 3 substituents selected from
   (1) a C₁₋₆ alkyl group and
   (2) a phenyl group optionally substituted by 1 to 3 substituents selected from a halogen atom, a C₁₋₆ alkyl group optionally substituted by 1 to 3 halogen atoms and a C₁₋₆ alkoxy group or
(B) a piperidinyl group optionally substituted by 1 to 3 pyrimidinyl groups; and
R³ is a C₁₋₆ alkyl group or a di-C₁₋₆ alkylamino group,
or a pharmaceutically acceptable salt thereof.

In some embodiments, the orexin type 2 receptor agonist is selected from Methyl (2R,3S)-3-((methylsulfonyl)amino)-2-(((cis-4-phenylcyclohexyl)oxy)methyl) piperidine-1-carboxylate; N-((2R,3S)-1-glycoloyl-2-(((cis-4-(2,3,6-trifluorophenyl)cyclohexyl)oxy)methyl) piperidin-3-yl) methanesulfonamide; and (2R,3S)—N-ethyl-2-(((cis-4-isopropylcyclohexyl)oxy)methyl)-3-((methylsulfonyl)amino) piperidine-1-carboxamide; or a pharmaceutically acceptable salt thereof.

In some embodiments, the orexin type 2 receptor agonist is selected from methyl (2R,3S)-3-((methylsulfonyl)amino)-2-(((cis-4-phenylcyclohexyl)oxy)methyl) piperidine-1-carboxylate, or a pharmaceutically acceptable salt thereof. Methyl(2R,3S)-3-((methylsulfonyl)amino)-2-(((cis-4-phenylcyclohexyl)oxy)methyl) piperidine-1-carboxylate is also referred to herein as Compound A.

In some embodiments, the orexin type 2 receptor agonist is selected from N-((2S,3S)-1-(2-hydroxy-2-methylpropanoyl)-2-((2,3′,5′-trifluorobiphenyl-3-yl)methyl) pyrrolidin-3-yl) methanesulfonamide; N-((2S,3S)-2-((2,3′-difluorobiphenyl-3-yl)methyl)-1-(2-hydroxy-2-methylpropanoyl) pyrrolidin-3-yl) ethanesulfonamide; methyl (2R,3S)-3-((methylsulfonyl)amino)-2-(((cis-4-phenylcyclohexyl)oxy)methyl) piperidine-1-carboxylate; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}methanesulfonamide; 4-(5-cyclopropyl-1,2,4-oxadiazol-3-yl)-N-{(1R,6S)-2,2-difluoro-6-[4-(propan-2-yl) piperazin-1-yl]cyclohexyl}-4-methylpiperidine-1-carboxamide; N-{(1R,6S)-2,2-difluoro-6-[4-(propan-2-yl) piperazin-1-yl]cyclohexyl}-4-{5-[(1S,2S)-2-fluorocyclopropyl]-1,2,4-oxadiazol-3-yl}-4-methylpiperidine-1-carboxamide; (2R)-2-cyclopropyl-2-{(1R,3S,5S)-3-[(3S,4R)-1-(5-fluoropyrimidin-2-yl)-3-methoxypiperidin-4-yl]-8-azabicyclo[3.2.1]octan-8-yl}acetamide; (R)-2-((1R,3S,5S)-3-((3S,4R)-1-(5-fluoropyrimidin-2-yl)-3-methoxypiperidin-4-yl)-8-azabicyclo[3.2.1]octan-8-yl)-3-methylbutaneamide; (R)-2-((1R,3S,5S)-3-((3S,4R)-1-(5-chloropyrimidin-2-yl)-3-ethoxypiperidin-4-yl)-8-azabicyclo[3.2.1]octan-8-yl)-2-cyclopropyl acetamide; (R)-2-cyclopropyl-2-((1R,3S,5S)-3-((2S,4S)-1-(5-fluoropyrimidin-2-yl)-2-methylpiperidin-4-yl)-8-azabicyclo[3.2.1]octan-8-yl) acetamide; and N-((2¹S,2⁴S,5²R,5³S)-6-oxo-3,8-dioxa-1 (2,3)-pyrazina-5 (2,1)-piperidina-2 (1,4)-cyclohexanacyclooctaphane-5³-yl) methanesulfonamide; or a pharmaceutically acceptable salt thereof.

In some embodiments, the orexin type 2 receptor agonist is a compound represented by the formula (II):

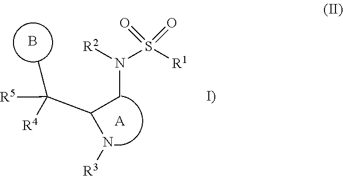

wherein
R¹ is
   (1) a C₁₋₆ alkyl group optionally substituted by 1 to 3 substituents selected from
      (a) a halogen atom, and
      (b) a C₁₋₆ alkoxy group,
   (2) a C₃₋₆ cycloalkyl group optionally substituted by 1 to 3 halogen atoms, or
   (3) a mono- or di-C₁₋₆ alkylamino group;
R² is a hydrogen atom;
R³ is
   (1) a C₁₋₆ alkoxy-carbonyl group,
   (2) a C₁₋₆ alkyl-carbonyl group optionally substituted by 1 to 3 hydroxy groups,
   (3) a mono- or di-C₁₋₆ alkyl-carbamoyl group,
   (4) a N—C₁₋₆ alkyl-N—C₁₋₆ alkoxy-carbamoyl group,
   (5) a C₃₋₆ cycloalkyl-carbonyl group (the C₃₋₆ cycloalkyl in the C₃₋₆ cycloalkyl-carbonyl group may be a bridged ring group) optionally substituted by 1 to 3 substituents selected from
      (a) a halogen atom,
      (b) a C₁₋₆ alkyl group optionally substituted by 1 to 3 halogen atoms,
      (c) a hydroxy group,
      (d) a C₁₋₆ alkoxy group, and
      (e) a cyano group,
   (6) an oxetanylcarbonyl group,
   (7) an azetidinylcarbonyl group optionally substituted by 1 to 3 substituents selected from
      (a) a halogen atom, and
      (b) a C₁₋₆ alkyl group, or
   (8) a 5-azaspiro[2.3]hexylcarbonyl group;
R⁴ and R⁵ are both hydrogen atoms;
Ring A is
   (1) a pyrrolidine ring, or
   (2) a piperidine ring; and
Ring B is
   (1) a benzene ring further substituted by one phenyl group optionally substituted by 1 to 3 substituents selected from
      (i) a halogen atom, and
      (ii) a C₁₋₆ alkyl group, and optionally further substituted by one halogen atom,
   (2) a pyridine ring further substituted by one phenyl group optionally substituted by 1 to 3 halogen atoms,
   (3) a thiazole ring further substituted by one phenyl group optionally substituted by 1 to 3 halogen atoms, or
   (4) a piperidine ring further substituted by one phenyl group;
or a pharmaceutically acceptable salt thereof.

In some embodiments, the orexin type 2 receptor agonist is N-((2S,3S)-1-(2-hydroxy-2-methylpropanoyl)-2-((2,3′,5′-trifluorobiphenyl-3-yl)methyl) pyrrolidin-3-yl) methanesulfonamide, or a pharmaceutically acceptable salt thereof. N-((2S,3S)-1-(2-hydroxy-2-methylpropanoyl)-2-((2,3′,5′-trifluorobiphenyl-3-yl)methyl) pyrrolidin-3-yl) methanesulfonamide is also referred to herein as Compound B.

In some embodiments, the orexin type 2 receptor agonist is N-((2S,3S)-2-((2,3′-difluorobiphenyl-3-yl)methyl)-1-(2-hydroxy-2-methylpropanoyl) pyrrolidin-3-yl) ethanesulfonamide, or a pharmaceutically acceptable salt thereof.

In some embodiments, the orexin type 2 receptor agonist is a compound represented by the formula (III):

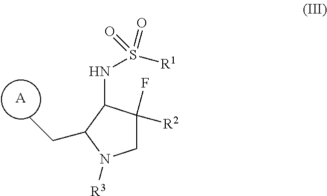

wherein
R¹ is
   (1) a C₁₋₆ alkyl group,
   (2) a mono- or di-C₁₋₆ alkylamino group, or
   (3) a C₃₋₆ cycloalkyl group;
R² is
   (1) a hydrogen atom,
   (2) a fluorine atom, or
   (3) a C₁₋₆ alkyl group;
R³ is
   (1) a C₁₋₆ alkyl-carbonyl group optionally substituted by 1 to 3 substituents selected from
      (a) a halogen atom,
      (b) a hydroxy group, and
      (c) a cyano group,
   (2) a C₁₋₆ alkoxy-carbonyl group,
   (3) a C₃₋₁₀ cycloalkyl-carbonyl group (the C₃₋₁₀ cycloalkyl moiety of the C₃₋₁₀ cycloalkyl-carbonyl group is optionally bridged) optionally substituted by 1 to 3 substituents selected from
      (a) a halogen atom,
      (b) a hydroxy group,
      (c) a cyano group, and
      (d) a C₁₋₆ alkyl group,
   (4) a 3- to 14-membered non-aromatic heterocyclylcarbonyl group optionally substituted by 1 to 3 substituents selected from
      (a) a halogen atom,
      (b) a hydroxy group, and
      (c) a C₁₋₆ alkyl group,
   (5) a mono- or di-C₁₋₆ alkyl-carbamoyl group, or
   (6) a N—C₁₋₆ alkyl-N—C₁₋₆ alkoxy-carbamoyl group; and
Ring A is
   (1) a benzene ring optionally substituted by one substituent selected from
      (a) a C₆₋₁₄ aryl group optionally substituted by 1 to 3 substituents selected from
         (i) a halogen atom,
         (ii) an optionally halogenated C₁₋₆ alkyl group, and
         (iii) an optionally halogenated C₁₋₆ alkoxy group, and
      (b) a 5- to 14-membered aromatic heterocyclic group optionally substituted by 1 to 3 substituents selected from
         (i) a C₁₋₆ alkyl group, and
         (ii) a C₁₋₆ alkoxy group, and optionally further substituted by 1 to 3 halogen atoms, or
   (2) a 5- or 6-membered aromatic heterocycle further substituted by one C₆₋₁₄ aryl group
optionally substituted by 1 to 3 halogen atoms;
or a pharmaceutically acceptable salt thereof.

In some embodiments, the orexin type 2 receptor agonist is N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}ethanesulfonamide, or a pharmaceutically acceptable salt thereof.

In some embodiments, the orexin type 2 receptor agonist is N-((2S,3R)-4,4-difluoro-1-(2-hydroxy-2-methylpropanoyl)-2-((2,3′,5′-trifluoro-[1,1′-biphenyl]-3-yl)methyl) pyrrolidin-3-yl) methanesulfonamide, or a pharmaceutically acceptable salt thereof. N-((2S,3R)-4,4-difluoro-1-(2-hydroxy-2-methylpropanoyl)-2-((2,3′,5′-trifluoro-[1,1′-biphenyl]-3-yl)methyl) pyrrolidin-3-yl) methanesulfonamide is also referred to herein as Compound C.

In some embodiments, the orexin type 2 receptor agonist is selected from N′-{(2S,3R,4S)-1-(azetidine-1-carbonyl)-4-fluoro-2-[(2-fluoro-3-methyl[1,1′-biphenyl]-3-yl)methyl]pyrrolidin-3-yl}-N,N-dimethyl sulfuric diamide; N-[(2S,3R)-2-[(2,3′-difluoro[1,1′-biphenyl]-3-yl)methyl]-4,4-difluoro-1-(2-methylpropanoyl) pyrrolidin-3-yl]ethanesulfonamide; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}ethanesulfonamide; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}methanesulfonamide; N-{(2S,3R)-1-(bicyclo[1.1.1]pentane-1-carbonyl)-4,4-difluoro-2-[(2,3′,5′-trifluoro[1,1′-biphenyl]-3-yl)methyl]pyrrolidin-3-yl}methanesulfonamide; N-{(2S,3R)-1-(cyclopropanecarbonyl)-4,4-difluoro-2-[(2,3′,5′-trifluoro[1,1′-biphenyl]-3-yl)methyl]pyrrolidin-3-yl}ethanesulfonamide; N-{(2S,3R)-4,4-difluoro-1-((1S,3R)-3-fluorocyclobutane-1-carbonyl)-2-[(2,3′,5′-trifluoro[1,1′-biphenyl]-3-yl)methyl]pyrrolidin-3-yl}ethanesulfonamide; N-{(2S,3R)-4,4-difluoro-1-((1S,3R)-3-fluorocyclobutane-1-carbonyl)-2-[(2,3′,5′-trifluoro[1,1′-biphenyl]-3-yl)methyl]pyrrolidin-3-yl}methanesulfonamide; N′-{(2S,3R)-1-(azetidine-1-carbonyl)-4,4-difluoro-2-[(2-fluoro-3′-methyl[1,1′-biphenyl]-3-yl)methyl]pyrrolidin-3-yl}-N,N-dimethylsulfuric diamide; or a pharmaceutically acceptable salt thereof.

One useful OX2R agonist is N-((2S,3S)-1-(2-hydroxy-2-methylpropanoyl)-2-((2,3′,5′-trifluorobiphenyl-3-yl)methyl) pyrrolidine-3-yl) methanesulfonamide and pharmaceutically acceptable salts and hydrates thereof. This compound is described in WO 2019/027058.

Another useful OX2R agonist is methyl(2R,3S)-3-((methylsulfonyl)amino)-2-(((cis-4-phenylcyclohexyl)oxy)methyl) piperidine-1-carboxylate and pharmaceutically acceptable salts and hydrates thereof. This compound is described in WO2017/135306.

Another useful OX2R agonist is N-((2S,3S)-2-((2,3′-difluorobiphenyl-3-yl)methyl)-1-(2-hydroxy-2-methylypropanoyl) pyrrolidin-3-yl) ethanesulfonamide and pharmaceutically acceptable salts and hydrates thereof. This compound is described in WO 2019/027058.

Another useful OX2R agonist is N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3 -yl}methanesulfonamide and pharmaceutically acceptable salts and hydrates thereof. This compound is described in WO2020/158958.

Further useful OX2R agonists include JZP441/DSP-0187, ALKS2680, and E2086.

Useful OX2R agonists are also described in PCT Published Appl. No. WO2021/107023, which is fully incorporated by reference herein, and include the following compounds and their pharmaceutically acceptable salts and hydrates: 4-(5-cyclopropyl-1,2,4-oxadiazol-3-yl)-N-{(1R,6S)-2,2-difluoro-6-[4-(propan-2-yl) piperazin-1-yl]cyclohexyl}-4-methylpiperidine-1-carboxamide represented by the following formula (IV):

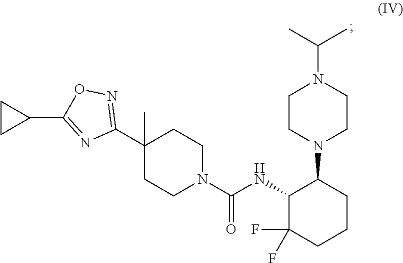 [SMILES: CC(C)N1CCN([C@H]2CCCC(F)(F)[C@@H]2NC(=O)N2CCC(C)(c3noc(C4CC4)n3)CC2)CC1]

and
N-{(1R,6S)-2,2-difluoro-6-[4-(propan-2-yl) piperazin-1-yl]cyclohexyl}-4-{5-[(1S,2S)-2-fluorocyclopropyl]-1,2,4-oxadiazol-3-yl}-4-methylpiperidine-1-carboxamide represented by the following formula (V):

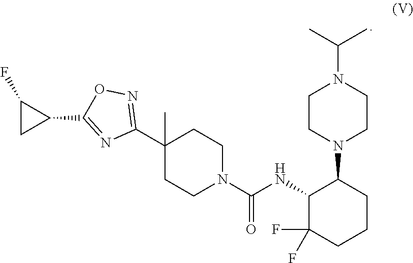 [SMILES: CC(C)N1CCN([C@H]2CCCC(F)(F)[C@@H]2NC(=O)N2CCC(C)(c3noc([C@@H]4C[C@@H]4F)n3)CC2)CC1]

Useful OX2R agonists are described in PCT Published Appl. No. WO2022/014680, which is fully incorporated by reference herein, and include the following compounds and their pharmaceutically acceptable salts and hydrates: (2R)-2-cyclopropyl-2-{(1R,3S,5S)-3-[(3S,4R)-1-(5-fluoropyrimidin-2-yl)-3-methoxypiperidin-4-yl]-8-azabicyclo[3.2.1]octan-8-yl}acetamide represented by the following formula (VI):

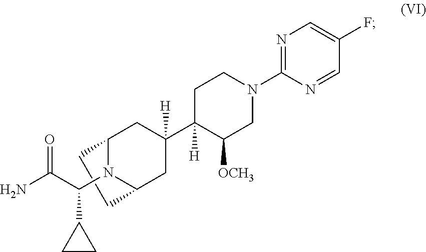 [SMILES: CO[C@@H]1CN(c2ncc(F)cn2)CC[C@@H]1[C@@H]1C[C@H]2CC[C@@H](C1)N2[C@@H](C(N)=O)C1CC1]

(R)-2-((1R,3S,5S)-3-((3S,4R)-1-(5-fluoropyrimidin-2-yl)-3-methoxypiperidin-4-yl)-8-azabicyclo[3.2.1]octan-8-yl)-3-methylbutaneamide represented by the following formula (VII):

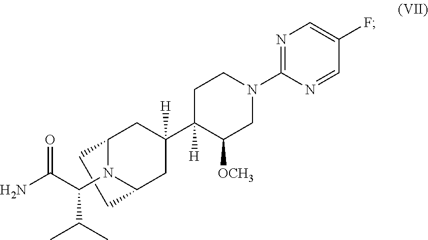 [SMILES: CO[C@@H]1CN(c2ncc(F)cn2)CC[C@@H]1[C@@H]1C[C@H]2CC[C@@H](C1)N2[C@@H](C(N)=O)C(C)C]

(R)-2-((1R,3S,5S)-3-((3S,4R)-1-(5-chloropyrimidin-2-yl)-3-ethoxypiperidin-4-yl)-8-azabicyclo[3.2.1]octan-8-yl)-2-cyclopropyl acetamide represented by the following formula (VIII):

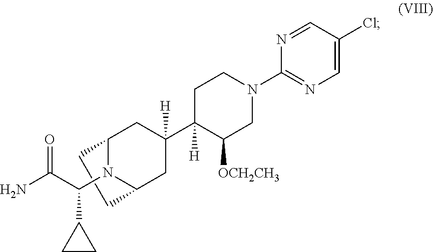 [SMILES: CCO[C@@H]1CN(c2ncc(Cl)cn2)CC[C@@H]1[C@@H]1C[C@H]2CC[C@@H](C1)N2[C@@H](C(N)=O)C1CC1]

and
(R)-2-cyclopropyl-2-((1R,3S,5S)-3-((2S,4S)-1-(5-fluoropyrimidin-2-yl)-2-methylpiperidin-4-yl)-8-azabicyclo[3.2.1]octan-8-yl) acetamide represented by the following formula (IX):

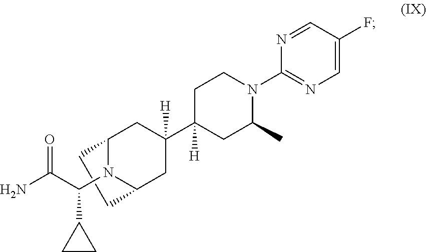 [SMILES: C[C@H]1C[C@@H]([C@@H]2C[C@H]3CC[C@@H](C2)N3[C@@H](C(N)=O)C2CC2)CCN1c1ncc(F)cn1]

Useful OX2R agonists are described in PCT Published Appl. Nos. WO2020/167701, WO2020/167706, WO2021/026047, WO2022/040070, WO2022/040058, WO2022/109117, WO 2022119888, WO 2022/132696 and WO 2022094012, which are fully incorporated by reference herein.

Useful OX2R agonists are described in PCT Published Appl. No. WO2022/051583 and No. WO2022/051596, which are fully incorporated by reference herein.

Useful OX2R agonists are described in PCT Published Appl. No. WO2021/108628, which is fully incorporated by reference herein, and include the following compounds and their pharmaceutically acceptable salts and hydrates thereof represented by Formula I-A:

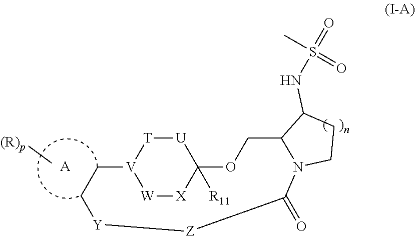

wherein: ring A is selected from the group consisting of phenyl, pyridinyl, pyridazinyl, pyrimidinyl, pyrazinyl, and triazinyl; n is 1, 2, or 3;
   T is CR₁R₂ or O;
   W is CR₄R₅ or O;
   U is CR₆R₇;
   X is CR₈R₉;
   V is CR₃ or N;
   Y is NR₁₀, O or absent;
   Z is (CR₁₂R₁₃)ₘ;
R is halogen or deuterium; p is 0, 1, 2, 3, or 4; and m is 1, 2, 3, or 4;
   R₁, R₂, R₄, and R₅ are each, independently, selected from the group consisting of H, halogen, and deuterium; or, alternatively, R₂ and R₅ together with the carbon atoms to which they are attached, form a single bond;
   R₃ is selected from the group consisting of H, deuterium, halogen, hydroxyl, and cyano; or, alternatively, R₃ and R₁, together with the carbon atoms to which they are attached, form a C₃-C₅cycloalkyl; or, alternatively, R₃ and R₄, together with the carbon atoms to which they are attached, form a C₃-C₅ cycloalkyl;
   R₆, R₇, R₈, R₉, and R₁₁ are each, independently, selected from the group consisting of H, halogen, and deuterium;
   R₁₀ is selected from the group consisting of H, unsubstituted C₁-C₃alkyl, and C₁-C₃alkyl substituted with one or more halogen atoms; and each R₁₂ and R₁₃ is, independently, selected from the group consisting of H, halogen, deuterium, unsubstituted C₁-C₃alkyl, and C1-C₃alkyl substituted with one or more halogen atoms; and
   R¹² and R¹³ are, independently, selected from the group consisting of H, halogen, deuterium, unsubstituted C₁-C₃alkyl, and C₁-C₃alkyl substituted with one or more halogen atoms.

An example of a suitable OX2R agonist is N-((2¹S,2⁴S,5²R,5³S)-6-oxo-3,8-dioxa-1 (2,3)-pyrazina-5 (2,1)-piperidina-2 (1,4)-cyclohexanacyclooctaphane-5³-yl) methanesulfonamide represented by the following formula (X):

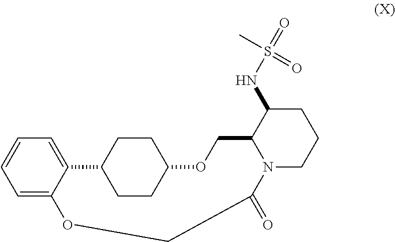

or a pharmaceutically acceptable salt or hydrate thereof.

The OX2R agonist may exist as a pharmaceutically acceptable salt. Examples of such salts include a salt with inorganic base, a salt with organic base, a salt with inorganic acid, a salt with organic acid, a salt with basic or acidic amino acid and the like. Examples of the salt with inorganic base include alkali metal salts such as sodium salt, potassium salt and the like, alkaline earth metal salts such as calcium salt, magnesium salt and the like, aluminum salt, ammonium salt and the like. Examples of the salt with organic base include salts with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine and the like. Examples of the salt with inorganic acid include salts with hydrochloric acid, hydrobromic acid, nitric acid, sulfuric acid, phosphoric acid and the like. Examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like. Examples of the salt with basic amino acid include salts with arginine, lysine, ornithine and the like. Examples of the salt with acidic amino acid include salts with aspartic acid, glutamic acid and the like.

The OX2R agonist may exist as a hydrate or a non-hydrate, or a non-solvate (e.g., anhydride), or a solvate (e.g., hydrate).

Furthermore, the OX2R agonist may exist as a pharmaceutically acceptable cocrystal or cocrystal salt. The cocrystal or cocrystal salt means a crystalline substance constituted with two or more special solids at room temperature, each having different physical properties (e.g., structure, melting point, melting heat, hygroscopicity, solubility and stability). The cocrystal or cocrystal salt may be produced by known methods.

### Pharmaceutical Compositions, Dosage Forms and Delivery Devices

The pharmaceutical composition comprises pharmaceutically acceptable carriers. As pharmaceutically acceptable carriers, various organic or inorganic carrier substances conventionally used as preparation materials can be used. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Examples of the dosage form of the aforementioned pharmaceutical composition include tablet (including sugar-coated tablet, film-coated tablet, orally disintegrating tablet), capsule (including soft capsule, microcapsule), granule, powder, troche, syrup, emulsion, suspension, films (e.g., orally disintegrable films), injection (e.g., subcutaneous injection, intravenous injection, intramuscular injection, intraperitoneal injection, drip infusion), external preparation (e.g., dermal preparation, ointment), suppository (e.g., rectal suppository, vaginal suppository), pellet, nasal preparation, pulmonary preparation (inhalant), eye drop and the like, which can be respectively safely administered orally or non-orally (e.g., topical, rectal, intravenous administration). These preparations may be a release control preparation such as an immediate-release preparation, a sustained-release preparation and the like.

In some embodiments, the pharmaceutical composition is formulated for oral administration. In some embodiments, the pharmaceutical composition is formulated for non-oral administration. In some embodiments, the pharmaceutical composition is formulated for intravenous administration, subcutaneous administration, transdermal administration, intradermal administration or transmucosal administration. In some embodiments, the pharmaceutical composition is formulated for intravenous administration. In some embodiments, the pharmaceutical composition is formulated for subcutaneous administration. In some embodiments, the pharmaceutical composition is formulated for transdermal administration. In some embodiments, the pharmaceutical composition is formulated for transmucosal administration.

### Sustained Release Formulation

In one embodiment, a pharmaceutical composition comprises (a) an orexin type 2 receptor (OX2R) agonist; and (b) one or more pharmaceutically acceptable carriers that are capable of providing a sustained release of the OX2R agonist at or below the maximum non-awakening plasma concentration.

In some embodiments, the pharmaceutical composition provides an average plasma concentration of the OX2R agonist after administration of about 0.01 ng/ml to about 1 mg/mL, about 0.03 ng/ml to about 300 ng/ml, about 0.05 ng/ml to about 100 ng/ml, about 0.08 ng/mL to about 50 ng/ml, or about 1 ng/ml to about 30 ng/mL.

### EXAMPLES

The following non-limiting examples provide those of ordinary skill in the art with a complete disclosure and description of how to make and use the compositions, and assay, screening, and therapeutic methods of the invention, and are not intended to limit the scope of what the inventors regard as their invention.

### Example 1: Effects of OX2R-Selective Agonists, Compound A and Compound C, on Respiratory Function Using OX-A and OX-B as Controls

### Methods

### Animals

For in vitro electrophysiologic recording, neonatal Sprague-Dawley (SD) rats obtained from Charles River Laboratories Italia (Lecco, Italy) were used at postnatal day 1-10 (P1-10). For in vivo studies, male SD rats obtained from Charles River Laboratories Japan (Kanagawa, Japan) were used for EMG recording of the diaphragm and genioglossus muscle at 7 weeks old. All adult rats and mice were housed under laboratory conditions (12 h light/dark cycles, lights on at 7:00) with food (CE-2, CLEA Japan Inc. for animals obtained in Japan; rat and mouse maintenance diet Altromin R, A. Rieper SpA, Bolzano, Italy for animals obtained in Italy) and water available ad libitum.

### Chemicals and Solutions

Compound A, methyl(2R,3S)-3-((methylsulfonyl)amino)-2-(((cis-4-phenylcyclohexyl)oxy)methyl) piperidine-1-carboxylate, Compound B, N-((2S,3S)-1-(2-hydroxy-2-methylpropanoyl)-2-((2,3′,5′-trifluorobiphenyl-3-yl)methyl) pyrrolidin-3-yl) methanesulfonamide, and Compound C, N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}methanesulfonamide, were synthesized by Takeda Pharmaceutical Company Limited. For in vitro studies, all drugs were dissolved in dimethyl sulfoxide (DMSO) and diluted in each experimental solution. For EMG study of the diaphragm and genioglossus muscle in rats, Compound C was solubilized in 0.1% (w/v) Polysorbate 80 and 20% (w/v) Captisol (CyDex Pharmaceuticals, KS, USA) solution and was administered intravenously in a volume of 1 mL/kg of body weight.

### Electrophysiologic Recording of Neurons in the Pre-Bötzinger Complex and Hypoglossal Motoneurons in Rat Medullary Slice

Neonatal rats (postnatal day 1-10) were decapitated and the neuraxis was isolated in a chamber filled with oxygenated artificial cerebrospinal fluid solution (ACSF, in mM: NaCl 125, KCl 2.5, MgCl2 1, CaCl2 2, NaHCO3 25, NaH2PO4 1.25, glucose 25, pH 7.4). The cerebellum was removed, and the brainstem was mounted in the vibratome (VT 1000S; Leica, Milan, Italy) chamber filled with oxygenated gluconate cutting solution (in mM: Kgluconate 130, KCl 15, EGTA 0.2, HEPES 20, glucose 25, kynurenic acid 2, pH 7.4). Transverse medullary slices containing the pre-Bötzinger complex and hypoglossal nucleus were cut (400-600 μm thick). The slices were transferred at room temperature for 1 min in an oxygenated mannitol cutting solution (in mM: D-mannitol 225, glucose 25, KCl 2.5, NaH2PO4 1.25, NaHCO3 26, CaCl2 0.8, MgCl2 8, kynurenic acid 2, pH 7.4). Slices were then transferred in ACSF solution at 30° C. for 30 min and at room temperature for 30 min. At least 30 min prior to the start of recording, each slice was transferred in the recording solution in which the concentration of K+ was raised to 9 mM and that of Mg2+ was decreased to 0.5 mM to ensure the production of long-term and stable rhythm. For the recording, the slice was transferred in a submerged recording chamber where it was continuously perfused with fresh extracellular modified ACSF solution (mACSF, in mM: NaCl 125, MgCl2 0.5, KCl 9, CaCl2 1, NaHCO3 25, NaH2PO4 1.25, glucose 25, pH 7.4) and maintained at 27±2° C., with a flow rate of 2.3±0.2 mL/min using a peristaltic pump. The pre-Bötzinger complex were located ventral to the nucleus ambiguous (J. C. Smith, H. H. Ellenberger, K. Ballanyi, D. W. Richter, J. L. Feldman, Pre-Bötzinger complex: a brainstem region that may generate respiratory rhythm in mammals, Science (New York, N.Y.) 254 (5032) (1991) 726-9). Hypoglossal motoneurons were identified based on their location within hypoglossal nucleus, characteristic size (20-60 μm in width) and shape (multipolar neurons with a non-branching axon projecting ventrolaterally towards the hypoglossal nerve rootlets), and physiology (50-200 MΩ in input resistance), as previously reported (G. D. Funk, J. J. Greer, The rhythmic, transverse medullary slice preparation in respiratory neurobiology: contributions and caveats, Respir Physiol Neurobiol 186 (2) (2013) 236-53; J. Q. Pilarski, H. E. Wakefield, A. J. Fuglevand, R. B. Levine, R. F. Fregosi, Increased nicotinic receptor desensitization in hypoglossal motor neurons following chronic developmental nicotine exposure, J Neurophysiol 107 (1) (2012) 257-64). Whole cell voltage-clamp recordings were carried out at a membrane potential of −60 mV and −55 mV for neurons in the pre-Bötzinger complex and hypoglossal motoneurons, respectively, using a borosilicate pipette filled with intracellular solution (in mM: Kgluconate 114, KCl 6, MgATP 4, NaGTP 0.3, Na-Phosphocreatine 10, HEPES 10, EGTA 0.2, pH 7.25 and osmolarity 300 mOsm with sucrose). Signals were acquired using Clampex 10.6 software (Axon, Molecular Devices, CA, USA) at a sampling rate of 10-20 kHz and filtered at 2-10 kHz using an 8-pole Bessel filter built within the software and analyzed using Clampfit 10.6 software (pClamp 10.6; Axon, Molecular Devices). Baseline and threshold levels for bursts were set using Clampfit software. Bursts were then automatically detected, and burst frequency was measured.

To evaluate the effects of vehicle (0.1% DMSO) on neuronal activities, after 5 min of stabilization, the neuronal activities under basal condition was recorded for 2-12 min, followed by recording in the presence of vehicle (0.1% DMSO) for 30 min. Mean values in burst frequency during the last 1-2 min of basal condition were used as control values, and those during the last 1-2 min of vehicle perfusion were used to calculate percent changes from the control values. As vehicle (0.1% DMSO) had no significant effects on burst frequency of neurons in the pre-Bötzinger complex and hypoglossal motoneurons, potential of drugs was assessed under vehicle (0.1% DMSO). To evaluate the effects of drugs on neuronal activities, after 5 min of stabilization, the neuronal activities were recorded in the presence of vehicle for 2-20 min followed by recording after the application of drugs at two to three increasing concentrations (each for 4-15 min) per neuron. Mean values in burst frequency during the last 1-2 min in the presence of vehicle was used as control values, and those during the last 1-2 min with stimulation by each concentration of drugs were used to calculate percent changes from control values.

### Electrophysiologic Recording of Phrenic Motoneurons in Rat Isolated Brainstem-Spinal Cord Preparations

Neonatal (postnatal day 1-4) rats were decapitated, and the brainstem-spinal cord was dissected in a bath containing ACSF. The cerebellum and pons were ablated. At least 30 min prior to the start of recording, the tissue was transferred in the recording solution in which the concentration of K+ was raised to 5 mM and that of Mg2+ was decreased to 0.5 mM to ensure production of long-term and stable rhythm (modified ACSF, in mM: NaCl 125, MgCl2 0.5, KCl 5, CaCl2 1, NaHCO3 25, NaH2PO4 1.25, glucose 30, pH 7.4). The tissues were glued on coverslips and transferred to a recording chamber and continuously perfused at 27±2° C. with mACSF at a flow rate of 2.3±0.2 mL/min. Activity of phrenic motoneurons was measured in electrophysiological recordings from C3-5 ventral root, outputs of phrenic motoneurons (J. J. Greer, J. C. Smith, J. L. Feldman, Respiratory and locomotor patterns generated in the fetal rat brain stem-spinal cord in vitro, J Neurophysiol 67 (4) (1992) 996-9; J. C. Smith, J. J. Greer, G. S. Liu, J. L. Feldman, Neural mechanisms generating respiratory pattern in mammalian brain stem-spinal cord in vitro. I. Spatiotemporal patterns of motor and medullary neuron activity, J Neurophysiol 64 (4) (1990) 1149-69), using a glass suction electrode. Signals were acquired in current-clamp configuration using Clampex 10.6 software (Axon, Molecular Devices) at a sampling rate of 10-20 kHz and filtered at 2-10 kHz using an 8-pole Bessel filter built within the software. Acquired signals were filtered with a bandpass filter (0.01-3000 Hz), rectified, and integrated with a time constant decay (50-200 ms) using LabChart software (AD Instruments, Dunedin, New Zealand). The rectification and integration was used for quantification of the activities of phrenic motoneurons because the spontaneous bursts detected in voltage signals are consisting of multiple positive and negative voltages from baseline (T. Sugita, et al.). Threshold levels for bursts were set using LabChart software (AD Instruments). Bursts were then automatically detected, and burst frequency was measured.

To evaluate the effects of vehicle (0.1% DMSO) on neuronal activities, after 5 min of stabilization, the neuronal activities under basal condition were recorded for 2-20 min, followed by recording with vehicle (0.1% DMSO) for 30-40 min. Mean values in burst frequency during the last 2 min of basal conditions were used as control values, and those during the last 2 min with vehicle perfusion were used to calculate percent changes from control values. As vehicle (0.1% DMSO) had no significant effects on burst frequency of phrenic neurons, potential of drugs was assessed under vehicle (0.1% DMSO). To evaluate the effects of drugs on neuronal activities, after 5 min of stabilization, the neuronal activities in the presence of vehicle were recorded for 5-30 min, followed by recording with drug perfusion for 30-40 min until plateau effect was achieved. Mean values in burst frequency during the last 2 min in the presence of vehicle was used as control values, and those during the last 2 min of drug perfusion were used to calculate percent changes from control values.

### EMG Recording of the Diaphragm in Anesthetized Rats

Rats were anaesthetized with isoflurane (induction: 2%, maintenance: 1.5%). Anesthetic depth was assessed by the reflex responses to paw pinches. The tail vein was cannulated for intravenous drug administration. Rats were laid on disposable body warmers to prevent decrease in body temperature. The diaphragm was exposed, then two needle electrodes were implanted into costal diaphragm to monitor diaphragm EMG. After 10 min of baseline recording, vehicle (0.1% [w/v] Polysorbate 80 and 20% [w/v] Captisol solution) or Compound C was administered intravenously, and then EMG was recorded for 10 min. EMG was amplified using Bio amp system (ML132, AD Instruments), high-pass filtered (10 Hz), digitized, and recorded using PowerLab software (AD Instruments), and then rectified and integrated with a time constant decay (100 ms) using LabChart software (AD Instruments). Threshold levels for bursts were set using LabChart software (AD Instruments). Bursts were then automatically detected, and burst frequency was measured. Mean values in burst frequency during 2 min before vehicle or Compound C administration were used as control values, and those during 10 min after vehicle or Compound C administration were used to calculate percent of control values.

### EMG Recording of the Genioglossus Muscle in Anesthetized Rats

EMG recording of the genioglossus muscle was conducted as described previously (G. H. Zhang, et al.) with slight modifications. Rats were anaesthetized with urethane (2.0 g/kg, intraperitoneal injection). Anesthetic depth was assessed by the reflex responses to paw pinches. The tail vein was cannulated for intravenous drug administration. Each rat was laid on a servo-controlled electric heating pad. The body temperature of the rat was monitored with a rectal probe of the pad and kept at around 37° C. Vagus nerves were sectioned bilaterally to increase genioglossus muscle activity (G. H. Zhang, et al.; G. M. Stettner, L. Kubin, Antagonism of orexin receptors in the posterior hypothalamus reduces hypoglossal and cardiorespiratory excitation from the perifornical hypothalamus, J Appl Physiol (1985) 114 (1) (2013) 119-30). Rats were tracheotomized, and the genioglossus muscle was exposed. Then two needle electrodes were implanted bilaterally into the genioglossus muscle to monitor EMG. After 10 min of baseline recording, vehicle (0.1% [w/v] Polysorbate 80 and 20% [w/v] Captisol solution) or Compound C was administered intravenously and then EMG was recorded for 10 min. EMG was amplified 1,000-fold, band-pass filtered (100-5,000 Hz) using Microelectrode AC Amplifier (Model 1800; A-M systems, Carlsborg, WA, USA), digitized using AD converter (Power 1401; Cambridge Electronic Design, Cambridge, UK), recorded using Spike2 software (Cambridge Electronic Design), and then rectified and integrated with a time constant decay (100 ms) using LabChart software (AD Instruments). Threshold levels for bursts were set using LabChart software (AD Instruments). Bursts were then automatically detected, and burst amplitude was measured. Mean values in burst amplitude during 2 min before drug administration were used as control values, and those during 10 min after vehicle or Compound C administration were used to calculate percent of control values.

### Results

Effects of Compound A, Compound B, and Compound C on the Activities of Neurons Associated with Respiratory Control

Effects of Compound A, Compound B, and Compound C on the activities of neurons in the pre-Bötzinger complex and hypoglossal motoneurons were investigated by measuring burst frequency detected by voltage clamp recording.

### Effects of Compound A, Compound B, and Compound C on the Activities of Neurons in the Pre-Bötzinger Complex in Rat Medullary Slices

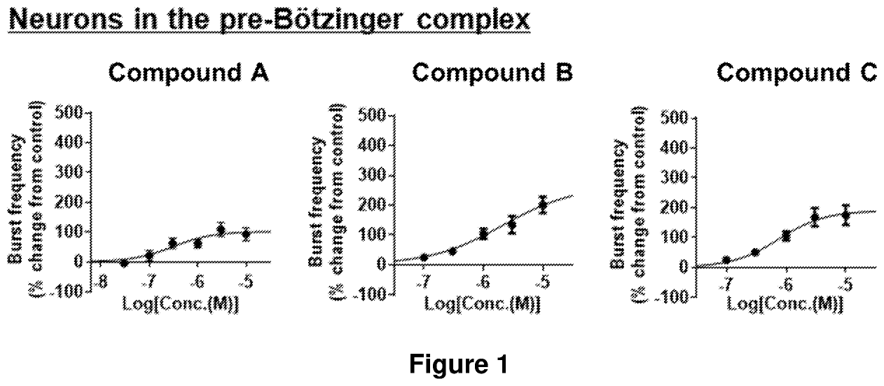

To examine the effects of Compound A, Compound B, and Compound C on the activities of neurons in the pre-Bötzinger complex, whole cell patch clamp recording using rat medullary slice was conducted. Compound A, Compound B, and Compound C all increased burst frequency in neurons in the pre-Bötzinger complex (FIG. 1). These results suggest that Compound A, Compound B, and Compound C increase the activities of neurons in the pre-Bötzinger complex in rat medullary slices.

Results are shown in FIG. 1, which shows how Compound A, Compound B, and Compound C activated neurons in the pre-Bötzinger complex in rat medullary slices.

### Effects of Compound A, Compound B, and Compound C on the Activities of Phrenic Motoneurons in Rat Isolated Brainstem-Spinal Cord Preparations

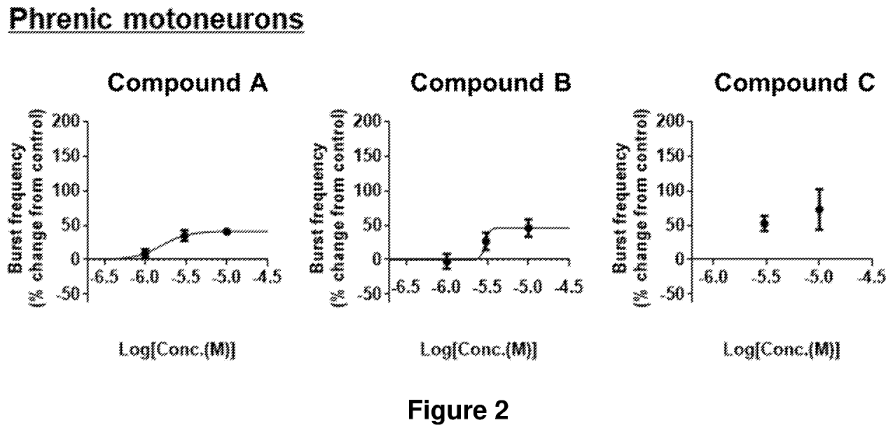

To examine the effects of Compound A, Compound B, and Compound C on the activities of phrenic motoneurons, electrophysiological recording using rat isolated brainstem-spinal cord preparations was conducted. Compound A, Compound B, and Compound C all increased burst frequency of phrenic motoneurons (FIG. 2). These results suggest that Compound A, Compound B, and Compound C increase activities of phrenic motoneurons in rat isolated brainstem-spinal cord preparations.

The results are shown in FIG. 2, which shows how Compound A, Compound B, and Compound C activated phrenic motoneurons in rat isolated brainstem-spinal cord preparations.

### Effects of Compound A, Compound B, and Compound C on the Activities of Hypoglossal Motoneurons in Rat Medullary Slices

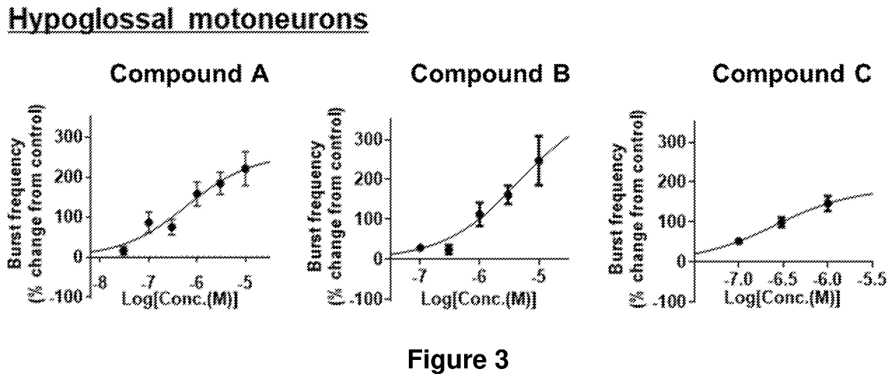

To examine the effects of Compound A, Compound B, and Compound C on the activities of hypoglossal motoneurons, whole cell patch clamp recording using rat medullary slices was conducted. Compound A, Compound B, and Compound C increased burst frequency (FIG. 3). These results suggest that Compound A, Compound B, and Compound C increase activities of hypoglossal motoneurons in rat medullary slices.

Results are shown in FIG. 3, which shows how Compound A, Compound B, and Compound C activated hypoglossal motoneurons in rat medullary slices.

### Effects of Compound C on the Activity of the Diaphragm in EMG Recording in Anesthetized Rats

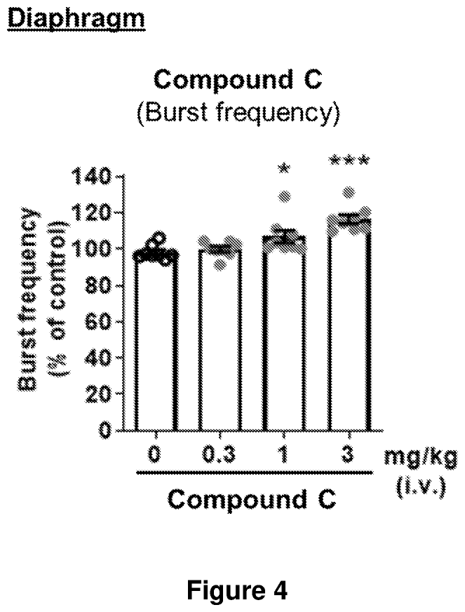

Due to technical feasibility of the experiment, anesthetized rats were used for EMG recording of the diaphragm. Compound C was administered intravenously to anesthetized rats in this experiment. To understand the relationship between the plasma effective concentration of Compound C for arousal and that for activation of the diaphragm, plasma concentration of Compound C after intravenous administration was measured. The maximum plasma concentrations of Compound C after intravenous administration at 0.3 and 1 mg/kg were similar to those after oral administration at 3 and 10 mg/kg, respectively. As oral administration of Compound C at 10 mg/kg or more produced potent arousal effects in rats, intravenous administration of Compound C at 0.3, 1 and 3 mg/kg was used to assess its potential to activate the diaphragm in EMG recording in anesthetized rats. As a result, intravenous administration of Compound C at 1 and 3 mg/kg significantly increased burst frequency of the diaphragm (FIG. 4). These results suggest that Compound C increases the activity of the diaphragm in anesthetized rats.

Results are shown in FIG. 4, which shows how intravenous administration of Compound C significantly increased burst frequency of the diaphragm in anesthetized rats.

### Effects of Compound C on the Activity of the Genioglossus Muscle in EMG Recording in Anesthetized Rats

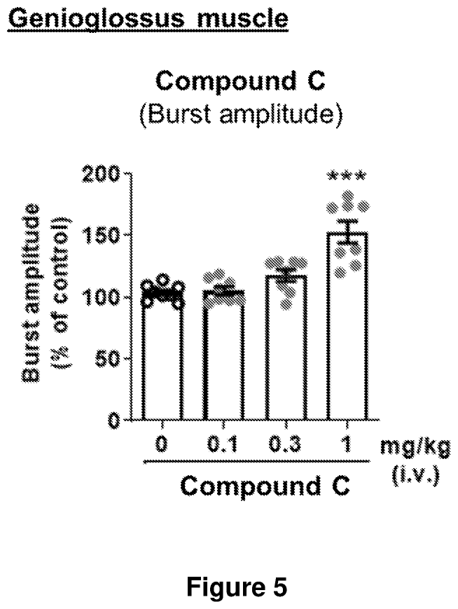

Effects of intravenous administration of Compound C on the activity of the genioglossus muscle in anesthetized rats were examined by EMG recording. Burst amplitude indicates the peak genioglossus muscle activity during burst. As a result, intravenous administration of Compound C at 1 mg/kg significantly increased burst amplitude (FIG. 5). These results suggest that Compound C increases the activity of the genioglossus muscle, especially in burst amplitude, in anesthetized rats.

FIG. 5 shows how intravenous administration of Compound C significantly increased burst amplitude of the genioglossus muscle in anesthetized rats.

Neurons in the pre-Bötzinger complex located in ventrolateral medulla are critical for the generation of inspiratory air flow. Additionally, they regulate activity of the diaphragm and genioglossus muscle through controlling the activities of phrenic motoneurons in cervical spinal cord and hypoglossal motoneurons in dorsomedial medulla, respectively (J. C. Smith, A. P. Abdala, A. Borgmann, I. A. Rybak, J. F. Paton, Brainstem respiratory networks: building blocks and microcircuits, Trends Neurosci 36 (3) (2013) 152-62)

Compound A, Compound B, and Compound C all increased the activities of neurons in the pre-Bötzinger complex and hypoglossal motoneurons in rat medullary slices. In these neurons, Compound A, Compound B, and Compound C all clearly increased burst frequency. OX2R protein may be expressed in pre-Bötzinger complex in rat brain slices (K. Mitsukawa, H. Kimura, Orexin 2 receptor (OX2R) protein distribution measured by autoradiography using radiolabeled OX2R-selective antagonist EMPA in rodent brain and peripheral tissues, Sci Rep 12 (1) (2022) 8473); therefore, it is conceivable that OX2R agonists directly stimulate neurons in the pre-Bötzinger complex. Compound A, Compound B, and Compound C also increased the activities of phrenic motoneurons, mainly by increasing burst frequency, in rat isolated brainstem spinal cord preparations.

Because the diaphragm and genioglossus muscle produce spontaneous burst during inspiration, burst frequency of these muscles would be related to respiratory rate of these mice. Thus, we see increased burst frequency in both the diaphragm and genioglossus muscle. However, in vivo studies revealed that Compound C increased burst frequency of the diaphragm, while it mainly increased burst amplitude of the genioglossus muscle in anesthetized rats. Our results suggest that OX2R agonists can modulate respiratory functions by increasing the activity of the diaphragm and genioglossus muscle through stimulation of neuronal network including neurons in the pre-Bötzinger complex, and phrenic and hypoglossal motoneurons in rodents.

As OX2R agonists activated the diaphragm and genioglossus muscle, they may have therapeutic potentials for both CSA and OSA.

OX2R agonists can modulate respiratory function by increasing the activity of the diaphragm and genioglossus muscle through stimulation of the neuronal network including neurons in the pre-Bötzinger complex, and phrenic and hypoglossal motoneurons.

### Example 2: A Clinical Trial to Evaluate the Safety, Tolerability, Pharmacodynamic, and Pharmacokinetic Effects of Overnight Intravenous Compound A in Patients with Obstructive Sleep Apnea

A phase 1b, randomized, double-blind, placebo-controlled, 3-period crossover study was conducted to evaluate the safety, tolerability, pharmacodynamic, and pharmacokinetic effects of overnight intravenous COMPOUND A in patients with obstructive sleep apnea (OSA). This study was planned to further evaluate COMPOUND A intravenous administration (IV) in adults with OSA, hypothesizing that nonwake-promoting exposures of COMPOUND A would increase genioglossal muscle tone and increase airway patency via the increasing activity of hypoglossal motoneurons. The main goal of this study was to evaluate the mechanistic potential of OX2R agonism for treatment of OSA using COMPOUND A IV infusion to determine appropriate use in future trials with an oral OX2R agonist.

Participants were male or female, aged 18 to 75 years inclusive, with OSA diagnosed according to the International Classification of Sleep Disorders-3 (ICSD-3) criteria and an apnea hypopnea index (AHI) of 12 to 50 events/hour of sleep. The study consisted of a screening period, 3 treatment periods separated by 2 washout periods, and a follow-up period. A total of 13 participants were enrolled and randomized, of which 12 (92.3%) participants completed all planned doses of study drug and the study. One (7.7%) participant did not complete all planned doses of study drug due to an adverse event and discontinued the study by withdrawal.

### Study Design

Eligible participants were randomly assigned (in a double-blinded manner) to 1 of 6 treatment sequences. In each treatment period, participants underwent 3 UACI assessments, a 10-minute evaluation that tested the key biological mechanism that causes OSA (as described by Osman, A. M., et al., 2019, Sleep, 42 (7), 1-10), followed by an overnight polysomnography (PSG). After the first baseline UACI assessment (U1), an IV infusion regimen of COMPOUND A or placebo was administered over approximately 10 hours to achieve specific plasma concentrations to evaluate the effect of COMPOUND A on the UACI and PSG metrics. Target plasma concentrations for the low-dose COMPOUND A IV regimen were 10 ng/mL for the second UACI (U2), 30 ng/ml for the third UACI (U3), and 10 ng/ml for the overnight PSG. Target plasma concentrations for the high-dose COMPOUND A IV regimen were 30 ng/mL for the U2, 90 ng/mL for the U3, and 30 ng/ml for the overnight PSG.

To achieve the target exposures for the UACI assessments (U2 and U3), there was a 20-minute loading period, followed by a 15-minute maintenance period for each. The overnight maintenance dose (approximately 490 minutes) covered the duration of a standard overnight PSG (approximately 8 hours). Doses were selected to cover the potential pharmacologically active concentration range for upper airway collapsibility. The concentration of COMPOUND A in solution was such that participants received the same volume of fluid in the low-dose COMPOUND A regimen, the high-dose COMPOUND A regimen, and placebo, allowing the study to be double-blinded and ensuring all participants were infused with the same volume of fluid.

On Day 1 of each treatment period, participants checked in approximately 3 hours prior to the scheduled dosing time. The procedure started in the evening to reduce the confounding factors of endogenous OX levels on assessments. Start time was adjusted based on the patient's usual bedtime. Following a physical examination, urine drug screen, and alcohol breathalyzer test, participants were fitted with an IV catheter for placebo/COMPOUND A infusion, electroencephalogram (EEG) electrodes to monitor wakefulness, choanal and epiglottic pressure sensors, and a nasal mask and pneumotachograph to measure airflow (Osman, et al. 2019).

Following the baseline UACI measurements, IV infusion of placebo or COMPOUND A began. COMPOUND A was tested at 2 concentrations in a stepwise manner, consisting of a 20 minute loading period followed by a 15 minute maintenance period per step. The UACI was tested at each concentration during the maintenance period. A Karolinska Sleepiness Scale (KSS) was administered immediately prior to each UACI testing period to gauge subjective sleepiness. After the U3 test, a KSS was administered, after which the UACI testing equipment (pressure sensors, genioglossus EMG electrodes) was removed with the exception of EEG electrodes that remained in place to record PSG throughout the evening. Prior to lights off, a final KSS was administered. Lights-off occurred, and participants were then allowed to sleep until the following morning (Day 2). Infusion of COMPOUND A/placebo was maintained throughout the night during the nocturnal PSG (nPSG) and ended approximately 8 hours after lights off. Participants were then discharged on Day 2. This procedure was repeated at each treatment period.

Safety assessments, including AEs, safety labs, vital signs, and ECGs were recorded at particular time points. Intervals between treatment periods were at least 48 hours and no more than 14 days from end of infusion to start of the next infusion. Seven (+2) days following discharge from the third treatment period, participants were contacted by telephone to review concomitant medications and AEs.

### Treatments and Dosing

In each treatment period, participants received either placebo or 1 of 2 dose regimens of COMPOUND A, each targeting a 3 fold range in COMPOUND A exposure (see Table 1).

Each dose regimen was designed to achieve target COMPOUND A plasma concentrations for U2, U3, and PSG: 10, 30, and 10 ng/ml in the COMPOUND A low dose regimen and 30, 90, and 30 ng/ml in the COMPOUND A high dose regimen. To achieve the target exposures for the UACI assessments (U2 and U3), there was a 20 minute loading period followed by a 15 minute maintenance period for each. The overnight maintenance dose (approximately 490 minutes) was to cover the duration of a standard overnight PSG (approximately 8 hours). Doses were selected to cover the potential pharmacologically active concentration range for upper airway collapsibility. The concentration of COMPOUND A in solution was such that participants received the same volume of fluid in the low dose COMPOUND A regimen, the high dose COMPOUND A regimen, and placebo.

TABLE 1
Dose and Regimen

| | Low-dose Regimen | | | High-dose Regimen | | |
|---|---|---|---|---|---|---|
| | Target | | | Target | | |
| | Plasma | Infusion | Total | Plasma | Infusion | Total |
| | Conc. | Rate | Dose | Conc. | Rate | Dose |
| | [ng/mL] | [mg/h] | [mg] | [ng/mL] | [mg/h] | [mg] |
| Level 1 loading (20 minutes) | 10 | 1.2 | 0.6 | 30 | 3.6 | 1.8 |
| Level 1 maintenance (15 minutes) | | 0.8 | | | 2.4 | |
| Level 2 loading (20 minutes) | 30 | 3 | 1.6 | 90 | 9 | 4.8 |
| Level 2 maintenance (15 minutes) | | 2.4 | | | 7.2 | |
| Pause infusion for 40 minutes | — | 0 | 0 | — | 0 | 0 |
| Overnight maintenance (490 | 10 | 0.43 | 3.5 | 30 | 1.3 | 10.5 |
| minutes) | | | | | | |

Conc: concentration

### Primary Endpoints

The primary endpoint was the number of participants with at least 1 treatment-emergent adverse event (TEAE).

### Secondary Endpoints

The secondary endpoints included change from baseline in upper airway collapsibility index (UACI) and AHI observed during overnight polysomnography metrics (PSGs).

### Results and Conclusions

COMPOUND A low-dose and high-dose regimens in adult participants with OSA were generally well tolerated with no safety concerns. Three (23.1%) participants experienced 4 drug-related TEAEs, all of which were mild in severity: 1 TEAE of blood pressure increased while on COMPOUND A low-dose regimen; 2 TEAEs of headache while on COMPOUND A low-dose regimen and placebo; and 1 TEAE of myalgia while on COMPOUND A low-dose regimen.

The TEAE of blood pressure increased led to study drug discontinuation during COMPOUND A low dose regimen. The participant was not exposed to placebo or COMPOUND A high dose regimen, which limits the possible interpretation of this event. The TEAE of drug related headache occurred on COMPOUND A low dose regimen and placebo, and no drug related TEAEs occurred on COMPOUND A high dose regimen, which discredit a possible relationship to COMPOUND A dose and minimizes the clinical significance of these drug related events.

No deaths or serious TEAEs were reported during the study.

All TEAEs were mild in intensity except for 1 moderate TEAE of headache (not related to the study drug nor the study procedure) reported on COMPOUND A low dose regimen. The most frequently reported TEAE was headache; no dose related trend was observed.

At predose, the placebo adjusted LS mean change in SBP/DBP was 2.44/0.29 mmHg and 9.01/1.84 mmHg for COMPOUND A low dose regimen and high dose regimen, respectively. The wide 95% CI for both SBP and DBP values at this time point reaffirm the high variability of the results. The absence of a clear trend in BP values is corroborated by the individual review of BP values in all participants. The clinical significance of these results is limited due to the variability of BP values at screening and predose, and inconsistent BP increases across treatment periods and time points.

There was a statistically significant and clinically significant decrease in AHI of approximately 9 events/hour on COMPOUND A high-dose regimen of 30 ng/ml overnight, with responders who had a larger decrease in AHI, as well as non-responders with no decrease in AHI. The LS mean was less than 30 events/hour, which translates into a reduction from severe to moderate OSA.

There was also a beneficial reduction in hypoxic burden at this dose, which can indicate a decrease in OSA severity on a measure affecting cardiovascular outcomes. Besides the statistical significance obtained with COMPOUND A high-dose regimen in the placebo-adjusted difference in the hypoxic burden LS means, this difference was also considered clinically significant for both low-dose and high-dose regimens as the hypoxic burden estimate was ≤53%*minutes/hour. This reduction in high hypoxic burden can be correlated with improved survival in OSA participants, as this parameter has been associated with cardiovascular risk determination for both fatal and non-fatal cardiovascular events (Azarbarzin, A., et al. 2019, European Heart Journal, 40 (14), 1149-1157; Martinez-Garcia, M. A., et al., 2023, Archivos de bronconeumologia, 59 (1), 36-43).

There was no consistent improvement in upper airway collapsibility as measured by UACI during wakefulness. In OSA endotypes, pharyngeal collapsibility during sleep appeared to decrease in measures of Vₚₐₛₛᵢᵥₑ and V_{active}, suggesting improvement in upper airway collapse during sleep with COMPOUND A high-dose regimen. Loop gain was also decreased at this dose, indicating stabilization of central ventilatory response, which can centrally reduce AHI. Arousal threshold in response to ventilatory disturbance was decreased, consistent with wake-promoting effects of COMPOUND A.

When administered continuously throughout the sleep period, COMPOUND A at 30 ng/ml significantly decreased REM and impacted sleep continuity by increasing wake after sleep onset and reducing sleep efficiency, although it did not cause an inability to fall asleep at this dose.

All the publications, patents, and the patent applications cited herein are incorporated herein by reference in their entireties.

The present disclosure is not to be limited in terms of the particular embodiments described in this application, which are intended as single illustrations of individual aspects of the disclosure. All the various embodiments of the present disclosure will not be described herein. Any modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1 to 3 cells refers to groups having 1, 2, or 3 cells. Similarly, a group having 1 to 5 cells refers to groups having 1, 2, 3, 4, or 5 cells, and so forth.

What is claimed is:

1. A method for improving respiratory function during sleep in a subject in need thereof, comprising administering to the subject an orexin type 2 receptor agonist.

2. The method of claim 1, wherein administering the orexin type 2 receptor agonist regulates inspiratory air flow in the subject.

3. The method of claim 1 or claim 2, wherein the orexin type 2 receptor agonist is administered in an amount that is effective to improve respiratory function while not providing an arousal response or wakefulness in the subject.

4. The method of claim 3, wherein the arousal response or wakefulness is determined by measuring sleep latency in one or more subjects diagnosed with sleep apnea.

5. The method of any of claims 1-3, wherein the subject has sleep apnea.

6. The method of claim 5, wherein the sleep apnea is selected from obstructive sleep apnea (OSA), treatment emergent sleep apnea, and central sleep apnea (CSA).

7. The method of claim 6, wherein the sleep apnea is obstructive sleep apnea (OSA).

8. The method of any one of claims 1-7, wherein administration of the OX2R agonist reduces the Apnea Hypopnea Index (AHI) in the subject.

9. The method of any one of claims 1-7, wherein administration of the OX2R agonist reduces the hypoxic burden in the subject.

10. The method of any one of claims 1-9, wherein the administration of the OX2R agonist provides a blood plasma concentration of the OX2R agonist at or below a maximum non-awakening plasma concentration of the agonist over a dosing interval.

11. The method of claim 10, wherein the blood plasma concentration of the OX2R agonist is selected by:
i) determining the non-awakening plasma concentration of the OX2R agonist that does not provide an arousal response or wakefulness in a human; and
ii) determining a dose of the OX2R agonist that will provide a blood plasma concentration which is at or below the maximum non-awakening plasma concentration.

12. The method of claim 10, wherein administration of the OX2R agonist provides a blood plasma concentration of the OX2R agonist after administration of about 1/50 to about 1/1 of the maximum non-awakening concentration of the OX2R agonist.

13. The method of claim 12, wherein the blood plasma concentration of the OX2R agonist is about 1/20 to about 1/1 of the maximum non-awakening concentration of the OX2R agonist.

14. The method of any one of claims 1-13, wherein the agonist is administered orally, intravenously, subcutaneously, transdermally, or transmucosally.

15. The method of claim 14, wherein the OX2R agonist is administered orally.

16. The method of any one of claims 1-15, wherein the OX2R agonist is administered once per day.

17. The method of any one of claims 1-15, wherein the OX2R agonist is administered from about 5 minutes to about 5 hours before the subject's bedtime.

18. The method of any of claims 1-17, wherein the orexin type 2 receptor agonist is a compound represented by the formula (I): 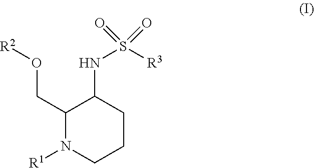
wherein
R¹ is
(1) a hydrogen atom,
(2) a C₁₋₆ alkyl-carbonyl group optionally substituted by 1 to 7 substituents selected from
(i) a halogen atom, (ii) a cyano group, (iii) a hydroxy group, (iv) a C₃₋₁₀ cycloalkyl group, (v) a C₁₋₆ alkoxy group, (vi) a C₆₋₁₄ aryl group, (vii) a C₆₋₁₄ aryloxy group, (viii) a pyrazolyl group, a thiazolyl group, a pyrimidinyl group or a pyridazinyl group, each of which is optionally substituted by an oxo group, (ix) a pyrazolyloxy group optionally substituted by 1 to 3 C₁₋₆ alkyl groups, (x) a C₁₋₆ alkyl-carbonyl group, (xi) a C₁₋₆ alkoxy-carbonyl group, (xii) a C₁₋₆ alkyl-carbonyloxy group, (xiii) a C₁₋₆ alkylsulfonyl group, (xiv) a mono- or di-C₁₋₆ alkylamino group, (xv) a C₁₋₆ alkyl-carbonylamino group and (xvi) a (C₁₋₆ alkyl) (C₁₋₆ alkyl-carbonyl)amino group,
(3) a C₃₋₁₀ cycloalkyl-carbonyl group optionally substituted by 1 to 3 substituents selected from a halogen atom, a cyano group, a hydroxy group, an oxo group and a C₁₋₆ alkyl group,
(4) a C₁₋₆ alkoxy-carbonyl group optionally substituted by 1 to 6 substituents selected from deuterium, a halogen atom and a C₆₋₁₄ aryl group,
(5) a C₃₋₁₀ cycloalkyloxy-carbonyl group optionally substituted by 1 to 3 substituents selected from a C₁₋₆ alkyl group,
(6) a C₆₋₁₄ aryl-carbonyl group optionally substituted by 1 to 3 substituents selected from a halogen atom and a C₆₋₁₄ aryl group,
(7) a C₆₋₁₄ aryloxy-carbonyl group,
(8) a furylcarbonyl group, a thienylcarbonyl group, a pyrazolylcarbonyl group, an isoxazolylcarbonyl group or a pyridylcarbonyl group, each of which is optionally substituted by 1 to 3 substituents selected from a C₁₋₆ alkyl group,
(9) an azetidinylcarbonyl group, an oxetanylcarbonyl group, a pyrrolidinylcarbonyl group, a tetrahydrofuranylcarbonyl group, a tetrahydropyranylcarbonyl group or a morpholinylcarbonyl group, each of which is optionally substituted by 1 to 3 substituents selected from an oxo group, a C₁-6 alkyl-carbonyl group, a C₁-6 alkoxy-carbonyl group and a C₁₋₆ alkylsulfonyl group,
(10) a mono- or di-C₁₋₆ alkyl-carbamoyl group optionally substituted by 1 to 3 substituents selected from a halogen atom, a cyano group, a hydroxy group and a C₁₋₆ alkoxy group,
(11) a mono- or di-C₃₋₁₀ cycloalkyl-carbamoyl group,
(12) a mono- or di-C₆₋₁₄ aryl-carbamoyl group,
(13) a C₁₋₆ alkylsulfonyl group,
(14) a C₃₋₁₀ cycloalkylsulfonyl group,
(15) a C₆₋₁₄ arylsulfonyl group optionally substituted by 1 to 3 halogen atoms,
(16) a thienylsulfonyl group, a pyrazolylsulfonyl group, an imidazolylsulfonyl group, a pyridylsulfonyl group or a dihydrochromenylsulfonyl group, each of which is optionally substituted by 1 to 3 substituents selected from a C₁₋₆ alkyl group,
(17) a mono- or di-C₁₋₆ alkyl-sulfamoyl group or
(18) a C₁₋₆ alkyl-carbonyl-carbonyl group;
R² is a C₃₋₆ cycloalkyl group, a pyrrolidinyl group, a piperidinyl group or a dioxanyl group, each of which is optionally substituted by 1 to 3 substituents selected from
(1) deuterium,
(2) a halogen atom,
(3) a hydroxy group,
(4) a C₁₋₆ alkyl group optionally substituted by 1 to 3 substituents selected from a halogen atom and a C₆₋₁₄ aryl group,
(5) a C₃₋₁₀ cycloalkyl group,
(6) a C₁₋₆ alkoxy group optionally substituted by a C₃₋₁₀ cycloalkyl group,
(7) a C₆₋₁₄ aryl group optionally substituted by 1 to 3 substituents selected from a halogen atom, a cyano group, a C₁-6 alkyl group optionally substituted by 1 to 3 halogen atoms, a C₁₋₆ alkoxy group optionally substituted by 1 to 3 halogen atoms and a hydroxy group,
(8) a C₆₋₁₄ aryloxy group,
(9) a tri-C₁₋₆ alkylsilyloxy group,
(10) a pyrazolyl group, a thiazolyl group, a pyridyl group, a pyrimidinyl group, a quinazolinyl group, a benzothiazolyl group or an isoquinolinyl group, each of which is optionally substituted by 1 to 3 substituents selected from a halogen atom, a C₁₋₆ alkyl group and a C₁₋₆ alkoxy group, and
(11) a C₆₋₁₄ aryl-carbonyl group; and
R³ is a C₁₋₆ alkyl group, or a mono- or di-C₁₋₆ alkylamino group,
or a pharmaceutically acceptable salt thereof.

19. The method of claim 18, wherein in formula (I):
R¹ is
(1) a hydrogen atom,
(2) a C₁₋₆ alkyl-carbonyl group optionally substituted by a hydroxy group,
(3) a cyclopropanecarbonyl group,
(4) a C₁₋₆ alkoxy-carbonyl group or
(5) a mono- or di-C₁₋₆ alkyl-carbamoyl group;
R² is
(A) a cyclohexyl group optionally substituted by 1 to 3 substituents selected from
(1) a C₁₋₆ alkyl group and
(2) a phenyl group optionally substituted by 1 to 3 substituents selected from a halogen atom, a C₁₋₆ alkyl group optionally substituted by 1 to 3 halogen atoms and a C₁₋₆ alkoxy group or
(B) a piperidinyl group optionally substituted by 1 to 3 pyrimidinyl groups; and
R³ is a C₁₋₆ alkyl group or a di-C₁₋₆ alkylamino group, or a pharmaceutically acceptable salt thereof.

20. The method of claim 18, wherein the orexin type 2 receptor agonist is selected from Methyl (2R,3S)-3-((methylsulfonyl)amino)-2-(((cis-4-phenylcyclohexyl)oxy)methyl) piperidine-1-carboxylate; N-((2R,3S)-1-glycoloyl-2-(((cis-4-(2,3,6-trifluorophenyl) cyclohexyl)oxy)methyl) piperidin-3-yl) methanesulfonamide; and (2R,3S)—N-ethyl-2-(((cis-4-isopropylcyclohexyl)oxy)methyl)-3-((methylsulfonyl)amino) piperidine-1-carboxamide; or a pharmaceutically acceptable salt thereof.

21. The method of claim 20, wherein the orexin type 2 receptor agonist is selected from methyl (2R,3S)-3-((methylsulfonyl)amino)-2-(((cis-4-phenylcyclohexyl)oxy)methyl) piperidine-1-carboxylate, or a pharmaceutically acceptable salt thereof.

22. The method of any of claims 1-17, wherein the orexin type 2 receptor agonist is selected from N-((2S,3S)-1-(2-hydroxy-2-methylpropanoyl)-2-((2,3′,5′-trifluorobiphenyl-3-yl)methyl) pyrrolidin-3-yl) methanesulfonamide; N-((2S,3S)-2-((2,3′-difluorobiphenyl-3-yl)methyl)-1-(2-hydroxy-2-methylpropanoyl) pyrrolidin-3-yl) ethanesulfonamide; methyl (2R,3S)-3-((methylsulfonyl)amino)-2-(((cis-4-phenylcyclohexyl)oxy)methyl) piperidine-1-carboxylate; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}methanesulfonamide; 4-(5-cyclopropyl-1,2,4-oxadiazol-3-yl)-N-{(1R,6S)-2,2-difluoro-6-[4-(propan-2-yl) piperazin-1-yl]cyclohexyl}-4-methylpiperidine-1-carboxamide; N-{(1R,6S)-2,2-difluoro-6-[4-(propan-2-yl) piperazin-1-yl]cyclohexyl}-4-{5-[(1S,2S)-2-fluorocyclopropyl]-1,2,4-oxadiazol-3-yl}-4-methylpiperidine-1-carboxamide; (2R)-2-cyclopropyl-2-{(1R,3S,5S)-3-[(3S,4R)-1-(5-fluoropyrimidin-2-yl)-3-methoxypiperidin-4-yl]-8-azabicyclo[3.2.1]octan-8-yl}acetamide; (R)-2-((1R,3S,5S)-3-((3S,4R)-1-(5-fluoropyrimidin-2-yl)-3-methoxypiperidin-4-yl)-8-azabicyclo[3.2.1]octan-8-yl)-3-methylbutaneamide; (R)-2-((1R,3S,5S)-3-((3S,4R)-1-(5-chloropyrimidin-2-yl)-3-ethoxypiperidin-4-yl)-8-azabicyclo[3.2.1]octan-8-yl)-2-cyclopropyl acetamide; (R)-2-cyclopropyl-2-((1R,3S,5S)-3-((2S,4S)-1-(5-fluoropyrimidin-2-yl)-2-methylpiperidin-4-yl)-8-azabicyclo[3.2.1]octan-8-yl) acetamide; and N-((2¹S,2⁴S,5²R,5³S)-6-oxo-3,8-dioxa-1 (2,3)-pyrazina-5 (2,1)-piperidina-2 (1,4)-cyclohexanacyclooctaphane-5³-yl) methanesulfonamide; or a pharmaceutically acceptable salt thereof.

23. The method of any of claims 1-17, wherein the orexin type 2 receptor agonist is a compound represented by the formula (II): 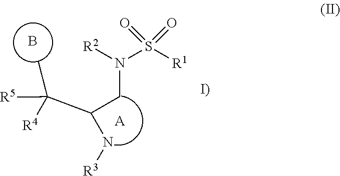
wherein
R¹ is
(1) a C₁₋₆ alkyl group optionally substituted by 1 to 3 substituents selected from
(a) a halogen atom, and
(b) a C₁₋₆ alkoxy group,
(2) a C₃₋₆ cycloalkyl group optionally substituted by 1 to 3 halogen atoms, or
(3) a mono- or di-C₁₋₆ alkylamino group;
R² is a hydrogen atom;
R³ is
(1) a C₁₋₆ alkoxy-carbonyl group,
(2) a C₁₋₆ alkyl-carbonyl group optionally substituted by 1 to 3 hydroxy groups,
(3) a mono- or di-C₁₋₆ alkyl-carbamoyl group,
(4) a N—C₁₋₆ alkyl-N—C₁₋₆ alkoxy-carbamoyl group,
(5) a C₃₋₆ cycloalkyl-carbonyl group (the C₃₋₆ cycloalkyl in the C₃₋₆ cycloalkyl-carbonyl group may be a bridged ring group) optionally substituted by 1 to 3 substituents selected from
(a) a halogen atom,
(b) a C₁₋₆ alkyl group optionally substituted by 1 to 3 halogen atoms,
(c) a hydroxy group,
(d) a C₁₋₆ alkoxy group, and
(e) a cyano group,
(6) an oxetanylcarbonyl group,
(7) an azetidinylcarbonyl group optionally substituted by 1 to 3 substituents selected from
(a) a halogen atom, and
(b) a C₁₋₆ alkyl group, or
(8) a 5-azaspiro[2.3]hexylcarbonyl group;
R⁴ and R⁵ are both hydrogen atoms;
Ring A is
(1) a pyrrolidine ring, or
(2) a piperidine ring; and
Ring B is
(1) a benzene ring further substituted by one phenyl group optionally substituted by 1 to 3 substituents selected from
(i) a halogen atom, and
(ii) a C₁₋₆ alkyl group, and
optionally further substituted by one halogen atom,
(2) a pyridine ring further substituted by one phenyl group optionally substituted by 1 to 3 halogen atoms,
(3) a thiazole ring further substituted by one phenyl group optionally substituted by 1 to 3 halogen atoms, or
(4) a piperidine ring further substituted by one phenyl group;
or a pharmaceutically acceptable salt thereof.

24. The method of claim 23, wherein the orexin type 2 receptor agonist is N-((2S,3S)-1-(2-hydroxy-2-methylpropanoyl)-2-((2,3′,5′-trifluorobiphenyl-3-yl)methyl) pyrrolidin-3-yl) methanesulfonamide, or a pharmaceutically acceptable salt thereof.

25. The method of claim 23, wherein the orexin type 2 receptor agonist is N-((2S,3S)-2-((2,3′-difluorobiphenyl-3-yl)methyl)-1-(2-hydroxy-2-methylpropanoyl) pyrrolidin-3-yl) ethanesulfonamide, or a pharmaceutically acceptable salt thereof.

26. The method of any of claims 1-17, wherein the orexin type 2 receptor agonist is a compound represented by the formula (III): 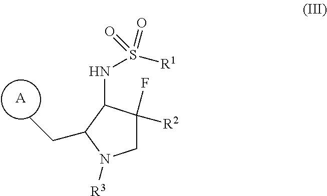
R¹ is
(1) a C₁₋₆ alkyl group,
(2) a mono- or di-C₁₋₆ alkylamino group, or
(3) a C₃₋₆ cycloalkyl group;
R² is
(1) a hydrogen atom,
(2) a fluorine atom, or
(3) a C₁₋₆ alkyl group;
R³ is
(1) a C₁₋₆ alkyl-carbonyl group optionally substituted by 1 to 3 substituents selected from
(a) a halogen atom,
(b) a hydroxy group, and
(c) a cyano group,
(2) a C₁₋₆ alkoxy-carbonyl group,
(3) a C₃₋₁₀ cycloalkyl-carbonyl group (the C₃₋₁₀ cycloalkyl moiety of the C₃₋₁₀ cycloalkyl-carbonyl group is optionally bridged) optionally substituted by 1 to 3 substituents selected from
(a) a halogen atom,
(b) a hydroxy group,
(c) a cyano group, and
(d) a C₁₋₆ alkyl group,
(4) a 3- to 14-membered non-aromatic heterocyclylcarbonyl group optionally substituted by 1 to 3 substituents selected from
(a) a halogen atom,
(b) a hydroxy group, and
(c) a C₁₋₆ alkyl group,
(5) a mono- or di-C₁₋₆ alkyl-carbamoyl group, or
(6) a N—C₁₋₆ alkyl-N—C₁₋₆ alkoxy-carbamoyl group; and
Ring A is
(1) a benzene ring optionally substituted by one substituent selected from
(a) a C₆₋₁₄ aryl group optionally substituted by 1 to 3 substituents selected from
(i) a halogen atom,
(ii) an optionally halogenated C₁₋₆ alkyl group, and
(iii) an optionally halogenated C₁₋₆ alkoxy group, and
(b) a 5- to 14-membered aromatic heterocyclic group optionally substituted by 1 to 3 substituents selected from
(i) a C₁₋₆ alkyl group, and
(ii) a C₁₋₆ alkoxy group, and
optionally further substituted by 1 to 3 halogen atoms, or
(2) a 5- or 6-membered aromatic heterocycle further substituted by one C₆₋₁₄ aryl group optionally substituted by 1 to 3 halogen atoms;
or a pharmaceutically acceptable salt thereof.

27. The method of claim 26, wherein the orexin type 2 receptor agonist is N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}ethanesulfonamide, or a pharmaceutically acceptable salt thereof.

28. The method of claim 26, wherein the orexin type 2 receptor agonist is N-((2S,3R)-4,4-difluoro-1-(2-hydroxy-2-methylpropanoyl)-2-((2,3′,5′-trifluoro-[1,1′-biphenyl]-3-yl)methyl) pyrrolidin-3-yl) methanesulfonamide, or a pharmaceutically acceptable salt thereof.

29. The method of claim 26, wherein the orexin type 2 receptor agonist is selected from N′-{(2S,3R,4S)-1-(azetidine-1-carbonyl)-4-fluoro-2-[(2-fluoro-3 methyl[1,1′-biphenyl]-3-yl)methyl]pyrrolidin-3-yl}-N,N-dimethyl sulfuric diamide; N-[(2S,3R)-2-[(2,3′-difluoro[1,1′-biphenyl]-3-yl)methyl]-4,4-difluoro-1-(2-methylpropanoyl) pyrrolidin-3-yl]ethanesulfonamide; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}ethanesulfonamide; N-{(2S,3R)-4,4-difluoro-1-(2-hydroxy-2-methylpropanoyl)-2-[(2,3′,5′-trifluoro[1,1′-biphenyl]-3-yl)methyl]pyrrolidin-3-yl}methanesulfonamide; N-{(2S,3R)-1-(bicyclo[1.1.1]pentane-1-carbonyl)-4,4-difluoro-2-[(2,3′,5′-trifluoro[1,1′-biphenyl]-3-yl)methyl]pyrrolidin-3-yl}methanesulfonamide; N-{(2S,3R)-1-(cyclopropanecarbonyl)-4,4-difluoro-2-[(2,3′,5′-trifluoro[1,1′-biphenyl]-3-yl)methyl]pyrrolidin-3-yl}ethanesulfonamide; N-{(2S,3R)-4,4-difluoro-1-((1S,3R)-3-fluorocyclobutane-1-carbonyl)-2-[(2,3′,5′-trifluoro[1,1′-biphenyl]-3-yl)methyl]pyrrolidin-3-yl}ethanesulfonamide; N-{(2S,3R)-4,4-difluoro-1-((1S,3R)-3-fluorocyclobutane-1-carbonyl)-2-[(2,3′,5′-trifluoro[1,1′-biphenyl]-3-yl)methyl]pyrrolidin-3-yl}methanesulfonamide; N′-{(2S,3R)-1-(azetidine-1-carbonyl)-4,4-difluoro-2-[(2-fluoro-3′-methyl[1,1′-biphenyl]-3-yl)methyl]pyrrolidin-3-yl}-N,N-dimethylsulfuric diamide; or a pharmaceutically acceptable salt thereof.

30. Use of an orexin type 2 receptor agonist for the manufacture of an medicament for improving respiratory function during sleep.

31. An orexin type 2 receptor agonist for use in the improvement of respiratory function during sleep.
